(19) United States
(12) Patent Application Publication
(10) Pub. No.: US 2024/0210409 A1
(43) Pub. Date: Jun. 27, 2024

(54) METHOD FOR DETECTING SUBSTANCE TO BE DETECTED WITHIN SAMPLE

(71) Applicant: NANOTIS CORPORATION, Tokyo (JP)

(72) Inventors: Jinsei MIYAZAKI, Osaka (JP); Yoko NAGAI, Chiba (JP); Teruyuki NAGAMUNE, Saitama (JP); Lisa Kato, Tokyo (JP)

(21) Appl. No.: 18/567,208

(22) PCT Filed: Oct. 7, 2020
(86) PCT No.: PCT/JP2020/038062

§ 371 Date: Jan. 18, 2024

(30) Foreign Application Priority Data
Oct. 7, 2019 (WO) PCT/JP2019/039460

(51) Int. Cl. G01N 33/58; G01N 21/64; G01N 33/569

(52) U.S. Cl. CPC G01N 33/582; G01N 21/6428; G01N 33/56983; G01N 2021/6432; G01N 2021/6439; G01N 2333/11

(57) ABSTRACT

A method for detecting a substance within a sample, including: locally concentrating a substance to be detected, a material for recognizing the substance to be detected, or a binding product of a substance to be detected and a material for recognizing the substance to be detected within a sample by electrophoresis or dielectrophoresis; optionally binding the substance to be detected and the material for recognizing the substance to be detected in the sample; measuring changes in fluorescence intensity of a binding product of the substance to be detected and the material for recognizing the substance to be detected; and confirming the presence of the substance to be detected in the sample based on the measured fluorescence intensity, in which the material for recognizing the substance to be detected is labeled with a fluorescent substance that emits fluorescence at a specific wavelength and specifically recognizes and binds to the substance.

### TECHNICAL FIELD

The present invention relates to a method for detecting a substance to be detected within a sample. Specifically, the present invention relates to a method capable of highly sensitive and fast detection for detecting a substance to be detected within a sample.

### BACKGROUND ART

A common feature of biological substances such as enzymes, nucleic acids, and antibodies is that they bind to their target substances with high specificity. So-called biosensing technology, which applies the properties of biological substances, has evolved at an accelerating pace, as disclosed in Non-Patent Literature 1 and 2, and has become widespread to the point where it is familiar to everyone.

As disclosed in Non-Patent Literature 1, various measurement methods using antibodies based on immunochromatography have been developed. Immunochromatography can be used regardless of knowledge, technique, equipment, or environment; therefore, for example, pregnancy testers using anti-human chorionic gonadotropin antibodies are widely used in households. Similarly, immunochromatography methods using antibodies that bind to influenza A and B viruses have contributed significantly to the rapid determination of influenza in clinical settings.

As described above, immunochromatography is a method that can easily be used to construct a system capable of convenient testing; however, there are technical hurdles in terms of improving sensitivity. For example, as disclosed in Non-Patent Literature 3, attempts have been made to improve sensitivity in immunochromatography by replacing labeled antibodies in the mobile phase with fluorescent substances or enzymes that cause luminescence.

In addition, regarding influenza viruses, as disclosed in Non-Patent Literature 4, the success in improving the sensitivity by 100 to 1000 times will make it possible to test specimens using saliva instead of nasal secretion. Therefore, the burden on medical professionals and patients is expected to be reduced.

As disclosed in Non-Patent Literature 5, recently, many attempts have been made to artificially create combinations of compounds called aptamers and the like, which can target and bind specifically to specific molecules.

Further, Non-Patent Literature 6 discloses a case in which bacteria in water were electrically concentrated, and the electrode system was used to measure the concentration of bacteria from impedance changes.

Furthermore, Non-Patent Literature 7 discloses that after concentrating bacteria near the electrode, antibodies specific to the bacteria are added to cause aggregation, followed by washing, and the bacteria are determined based on whether or not the antibodies caused the aggregation.

Patent Literature 1 discloses a microbial count/microbial concentration measurement device for measuring the number of microorganisms in a solution. Specifically, an antibody that targets Legionella bacteria and binds to them is fluorescently labeled, the Legionella bacteria are collected near the electrode by positive dielectrophoresis, and an optical fiber with the same size as the gap between the electrodes is used to observe how the fluorescence changes in the gap between the electrodes.

Patent Literature 2 discloses a method for separating a complex substance from molecules other than specific molecules contained in a sample. Specifically, AFP is used as a detection target substance, an anti-AFP antibody is bound to latex particles in advance, and antigen-antibody reaction is performed with fluorescently labeled Fab of antibody WA1 that binds to different epitopes of AFP. Then, dielectrophoresis is performed, and the dielectrophoresis with and without AFP is evaluated using fluorescently labeled Fabs subjected to sandwich reaction. In other words, in the case of technique disclosed in Patent Literature 2, by performing dielectrophoresis, the fluorescently labeled Fab bound to the latex particles via AFP is separated from the freely existing fluorescently labeled Fab; thus, the presence or absence of the antigen is distinguished by the presence or absence of dielectrophoresis.

### CITATION LIST

### Patent Literature

Patent Literature 1: Japanese Patent Application Laid-Open No. 2002-174636
Patent Literature 2: Japanese Patent Application Laid-Open No. 2001-165906

### Non-Patent Literature

Non-Patent Literature 1: Nishijima, SCANS NEWS 2005 II
Non-Patent Literature 2: C. Sebastian et al, Indo American Journal of Pharmaceutical Research. 2016:6 (07).
Non-Patent Literature 3: C. K. Lee et al, Journal of Clinical Virology 55 (2012) 239-243
Non-Patent Literature 4: A. Sueki et al, Clinica Chimica Acta 453, 71-74 (2016)
Non-Patent Literature 5: Taiichi Sakamoto, JOURNAL OF THE CRYSTALLOGRAPHIC SOCIETY OF JAPAN, Vol. 60, 129-134 (2018)
Non-Patent Literature 6: J. Suehiro et al 1999 J. Phys. D: Appl. Phys. 32 2814
Non-Patent Literature 7: J. Suehiro et al. J. Electrostatics 58 229 (2003)

### SUMMARY OF INVENTION

### Technical Problem

However, according to the method described in Non-Patent Literature 6, it is not only possible to identify bacteria but also to distinguish between bacteria and suspended particles.

In addition, according to the method described in Non-Patent Literature 7, the specificity of antibodies makes it possible to identify bacteria, however, the reaction cell must be larger than a certain size to ensure a sufficient flow rate, and the equipment also must be larger because it is necessary to switch between three types of liquids.

As described above, in order to concentrate a substance to be detected within a sample while maintaining the simplicity typified by immunochromatography, the problem is that the equipment becomes complicated and large. In addition, the biggest problem with immunochromatography is that it requires so-called B/F separation, which involves washing before and after the antigen-antibody reaction occurs.

An object of the present invention is to provide a novel measurement system that enables highly sensitive measurements without requiring specialized equipment, environment, knowledge, or technology.

### Solution to Problem

The present inventors have conducted extensive studies and thus thought concentrating a substance to be detected within a sample, as well as increasing the sensitivity of the detection unit, is one of the most rational methods to increase the sensitivity of the entire measurement system. The present inventors have further succeeded in combining the method with the technology of electrically concentrating the substance to be detected while based on a homogeneous detection system that does not require B/F separation. Moreover, the present inventors have conceived of measuring changes in the fluorescence intensity caused by the binding of the substance to be detected and a material for recognizing the substance to be detected in the present invention, thereby making it possible to provide a measurement system different from conventional methods. The present invention has created new value that combines sensitivity and simplicity.

The present invention is as follows.

[1] A method for detecting a substance to be detected within a sample, comprising:
a step of locally concentrating a substance to be detected, a material for recognizing the substance to be detected, or a binding product of a substance to be detected and a material for recognizing the substance to be detected within a sample by electrophoresis or dielectrophoresis;
a step of optionally binding the substance to be detected and the material for recognizing the substance to be detected in the sample;
a step of measuring changes in fluorescence intensity of a binding product of the substance to be detected and the material for recognizing the substance to be detected; and
a step of confirming the presence of the substance to be detected in the sample based on the measured fluorescence intensity,
wherein the material for recognizing the substance to be detected is labeled with a fluorescent substance that emits fluorescence at a specific wavelength and specifically recognizes and binds to the substance to be detected.

[2] The method according to [1], wherein the substance to be detected, the material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength, are mixed in the sample, thereby binding the substance to be detected and the material for recognizing the substance to be detected.

[2-1] A method for detecting a substance to be detected within a sample, comprising:
a step of locally concentrating a substance to be detected, a material for recognizing the substance to be detected, or a binding product of a substance to be detected and a material for recognizing the substance to be detected within a sample by electrophoresis or dielectrophoresis;
a step of optionally binding the substance to be detected and the material for recognizing the substance to be detected in the sample;
a step of measuring fluorescence intensity of a binding product of the substance to be detected and the material for recognizing the substance to be detected; and
a step of confirming the presence of the substance to be detected in the sample based on the measured fluorescence intensity, wherein
for fluorescence intensity measurement, a substance to be detected, a material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength, are mixed in a sample, thereby binding the substance to be detected and the material for recognizing the substance to be detected, and
the material for recognizing the substance to be detected is labeled with a fluorescent substance that emits fluorescence at a specific wavelength and specifically recognizes and binds to the substance to be detected.

[3] The method according to [1] or [2], wherein the substance to be detected, the material for recognizing the substance to be detected which is labeled with a fluorescent substance that emits fluorescence at a specific wavelength, and a substance to be detected or a substance other than the substance to be detected which is recognized at the same site as the substance to be detected, for the material for recognizing the substance to be detected, which is labeled with a fluorescent substance that fluoresces at a wavelength different from the specific wavelength or a quencher that absorbs fluorescence with the specific wavelength, are mixed in the sample, thereby binding the substance to be detected and the material for recognizing the substance to be detected in the sample.

[3-1] A method for detecting a substance to be detected within a sample, comprising:
a step of locally concentrating a substance to be detected, a material for recognizing the substance to be detected, or a binding product of a substance to be detected and a material for recognizing the substance to be detected within a sample by electrophoresis or dielectrophoresis;
a step of optionally binding the substance to be detected and the material for recognizing the substance to be detected in the sample;
a step of measuring fluorescence intensity of a binding product of the substance to be detected and the material for recognizing the substance to be detected; and
a step of confirming the presence of the substance to be detected in the sample based on the measured fluorescence intensity, wherein
for fluorescence intensity measurement, a substance to be detected, a material for recognizing the substance to be detected which is labeled with a fluorescent substance that emits fluorescence at a specific wavelength, and a substance to be detected or a substance other than the substance to be detected which is recognized at the same site as the substance to be detected, for the material for recognizing the substance to be detected, which is labeled with a fluorescent substance that fluoresces at a wavelength different from the specific wavelength or a quencher that absorbs fluorescence with the specific wavelength, are mixed in a sample, thereby binding the substance to be detected and the material for recognizing the substance to be detected in the sample, and
the material for recognizing the substance to be detected is labeled with a fluorescent substance that emits fluorescence at a specific wavelength and specifically recognizes and binds to the substance to be detected.

[4] The method according to any one of [1] to [3], wherein the material for recognizing the substance to be detected which is labeled with a fluorescent substance that emits a specific wavelength, and a material for recognizing the substance to be detected which is labeled with a fluorescent substance that fluoresces at a wavelength different from the specific wavelength or a quencher that absorbs fluorescence with the specific wavelength simultaneously, bind to the substance to be detected in the sample.

Note that regarding [2] or [3], [2] may be [2-1] and [3] may be [3-1].

[5] The method according to any one of [1] to [4], wherein the fluorescent substance that emits fluorescence at a specific wavelength is a fluorescent substance that is excited at wavelength 1 and emits wavelength 2, the fluorescent substance that fluoresces at a wavelength different from the specific wavelength is a fluorescent substance that absorbs wavelength 2 and emits wavelength 3, and excitation is induced at wavelength 1 so as to measure a fluorescence intensity of wavelength 3, and
wherein fluorescence intensity measurement of the wavelength 3 may be measurement of the wavelength 3 alone or both the wavelength 2 and the wavelength 3.

[6] The method according to any one of [1] to [5], wherein the fluorescent substance that emits fluorescence at a specific wavelength is a fluorescent substance that is excited at wavelength 1 and emits wavelength 2, the quencher that absorbs fluorescence with the specific wavelength is a fluorescent substance that absorbs wavelength 2 but does not emit fluorescent at least in a measurement wavelength range, and excitation is induced at wavelength 1 so as to measure a fluorescence intensity of wavelength 2.

[7] The method according to any one of [1] to [6], wherein any one selected from the group consisting of the material for recognizing the substance to be detected, the substance to be detected, and the substance other than the substance to be detected which is recognized at the same site as the substance to be detected, for the material for recognizing the substance to be detected, is physically or chemically bound to a carrier particle.

[8] The method according to any one of [1] to [7], wherein the material for recognizing the substance to be detected which is labeled with a fluorescent substance that emits a specific wavelength, and a material for recognizing the substance to be detected which is labeled with a fluorescent substance that fluoresces at a wavelength different from the specific wavelength or a quencher that absorbs fluorescence with the specific wavelength simultaneously, bind to the substance to be detected in the sample, and
wherein a material for recognizing the substance to be detected which is physically or chemically bound to the carrier particle, is further contained, and the substance to be detected and the material for recognizing the substance to be detected form a binding product in the sample.

[9] The method according to any one of [1] to [8], wherein the carrier particle is a metal fine particle, a metal oxide fine particle, a non-metal fine particle, a metal-coated resin fine particle, or a non-infectious spherical biological fine particle.

[10] The method according to any one of [1] to [9], wherein locally concentrating is carried out by positive dielectrophoresis.

[11] The method according to any one of [1] to [10], wherein the material for recognizing the substance to be detected is an antibody or an antibody fragment.

[12] The method according to any one of [1] to [11], wherein the substance to be detected is a bacterium, a virus, a nucleic acid, a protein, or a peptide.

[13] The method according to any one of [1] to [12], wherein the substance to be detected is an influenza-virus-derived substance.

[14] The method according to any one of [1] to [13], wherein at least one fluorescent substance is a quantum dot.

[15] The method according to any one of [1] to [14], wherein the substance to be detected is a nucleic acid, and the material for recognizing the substance to be detected is a nucleic acid complementary to at least part of the nucleic acid as the substance to be detected.

[16] The method according to any one of [1] to [15], wherein the substance to be detected is single-stranded RNA.

### Advantageous Effects of Invention

According to the present invention, highly sensitive measurements can be performed without requiring specialized equipment, environment, knowledge, and technology. In particular, according to the present invention, by measuring changes in the fluorescence intensity caused by the binding of a substance to be detected and a material for recognizing the substance to be detected, it becomes possible to provide a measurement system different from conventional methods.

### BRIEF DESCRIPTION OF DRAWINGS

FIG. 1 shows the fluorescence spectrum of Ab1Qd565.

FIG. 2 shows the absorption spectrum of NPQSY9.

FIG. 3 shows changes in the fluorescence spectrum of Ab1Qd565 due to the addition of NPQSY9.

FIG. 4 shows a typical configuration diagram of a detection cell.

FIG. 5 shows a typical configuration diagram of a detection device.

FIG. 6 shows the fluorescence spectrum of Ab1Qd655.

FIG. 7 shows the absorption spectrum of BSAQSY21.

FIG. 8 shows time-dependent changes in the fluorescence intensity of Ab1Qd655 due to the addition of BSAQSY21.

FIG. 9 shows the inhibition of quenching by the addition of BSA. This shows the results of fluorescence enhancement of Ab1Qd655 quenched by BSAQSY21.

FIG. 10 shows a comparison of fluorescence spectra (excitation at 370 nm) at each stage.

FIG. 11 shows a configuration diagram of a dielectrophoresis cell.

FIG. 12 shows a configuration diagram of a measurement unit.

FIG. 13 shows the absorption spectrum (dashed line) and fluorescence spectrum (solid line, excitation at 390 nm) of Ab2AT390.

FIG. 14 shows the absorption spectrum (dashed line) and fluorescence spectrum (solid line, excitation at 470 nm) of Ab3DY485.

FIG. 15 shows a configuration diagram of a detection cell.

FIG. 16 shows a configuration diagram of a test container. By lifting the swab 164 in the direction of the arrow, saliva is squeezed out and mixed with the reaction solution 162.

### DESCRIPTION OF EMBODIMENTS

Embodiments of the present invention will be described in detail below. The present invention is not limited to the following embodiments and can be implemented with various modifications within the scope of the gist of the present invention.

The detection method of the present invention is a method for detecting a substance to be detected within a sample, comprising:
a step of locally concentrating a substance to be detected, a material for recognizing the substance to be detected, or a binding product of a substance to be detected and a material for recognizing the substance to be detected within a sample by electrophoresis or dielectrophoresis;
a step of optionally binding the substance to be detected and the material for recognizing the substance to be detected in the sample;
a step of measuring changes in fluorescence intensity of a binding product of the substance to be detected and the material for recognizing the substance to be detected; and
a step of confirming the presence of the substance to be detected in the sample based on the measured fluorescence intensity,
wherein the material for recognizing the substance to be detected is labeled with a fluorescent substance that emits fluorescence at a specific wavelength and specifically recognizes and binds to the substance to be detected.

According to the detection method of the present invention, by carrying out each step described above, a substance to be detected can be detected by measuring changes in fluorescence spectra and the like using a fluorescent substance bound to a material for recognizing the substance to be detected. In addition, a method that allows electrophoresis or dielectrophoresis to be independently used for an application except for the measurement of fluorescence spectrum or the like, namely, the direct or indirect concentration of a substance to be detected, by detecting changes in fluorescence spectra and the like can be provided. Therefore, according to the present invention, a detection method capable of detection with higher sensitivity and speed than conventional methods using immunochromatography is provided.

In addition, in the detection method of the present invention, a substance to be detected may be detected by measuring changes in fluorescence spectra and the like using a fluorescent substance bound to a material for recognizing the substance to be detected while concentrating a binding product of the substance to be detected and the material for recognizing the substance to be detected, which specifically binds to the substance to be detected and is labeled with the fluorescent substance. In this case, the present invention also provides a detection method capable of detection with higher sensitivity and speed than conventional methods using immunochromatography.

In the present invention, regarding binding between a substance to be detected and a material for recognizing the substance to be detected, their concentrations, dissociation constant, binding curve, and the like are discussed. To detect a substance to be detected within a sample, it is considered that a drastic measure to increase sensitivity is to bring the concentration of the substance to be detected closer to the inflection point in the binding curve to increase the sensitivity of the system. Therefore, the present inventors thought that concentrating the specimen might lead to increased sensitivity, selected electrophoresis and dielectrophoresis as concentration methods, and conceived that dielectrophoresis is preferred. In consideration of a case in which a virus or the like is used as a substance to be detected, it is possible to use a virus as a substance to be detected and an antibody that binds to the surface antigen of the virus as a material for recognizing the substance to be detected; however, since the virus surface undergoes significant variation over time, a nucleoprotein may be used as a substance to be detected. In general, nucleoproteins can be extracted with surfactants. Since single-stranded RNA of influenza virus or the new coronavirus that caused the pandemic in 2020 (SARS-CoV-2) can be extracted under the same conditions as nucleoproteins, RNA may be a substance to be detected.

In the detection method of the present invention, a material for recognizing the substance to be detected, which specifically binds to a substance to be detected, is used. Preferably, a material for recognizing the substance to be detected, which can bind to a substance to be detected in water, is used. In this case, the material for recognizing the substance to be detected is in free form. As described later, a substance to be detected or a material for recognizing the substance to be detected, which specifically binds to a substance to be detected, may be physically or chemically bound to a carrier particle. In this case, the material for recognizing the substance to be detected is also in free form while being bound to the carrier particle but not immobilized on a substrate or the like. In other words, when the detection method of the present invention is carried out in a solution, a material for recognizing the substance to be detected moves in the solution.

When a material for recognizing the substance to be detected specifically binds to a substance to be detected, it means that the material for recognizing the substance to be detected has specificity to the substance to be detected as a subject for recognizing and binding. The term “specificity” used in the present invention refers to the binding of a material for recognizing the substance to be detected to a particular substance to be detected.

Examples of a sample as a subject to be measured in the detection method of the present invention include, but are not particularly limited to, samples from living organisms, samples collected from foods, and those present in buildings such as factories, schools, and hospitals.

In hospitals, nosocomial infections can be a problem, and by applying the detection method of the present invention, it is possible to detect the causative bacteria of nosocomial infections with high sensitivity and speed.

Examples of samples from living organisms include, but are not particularly limited to, mammals, specifically humans and livestock. Samples from living organisms such as saliva and blood may be used.

In the present invention, the above-described samples may be directly used as a detection target, or a sample diluted, suspended, or dissolved in a solvent such as water or alcohol may be used.

In addition, to detect the presence of a substance to be detected within a sample, it is preferable to use the sample as a sample for direct detection. However, before detection, the sample may be crushed by ultrasonication to elute the substance to be detected.

Examples of a substance to be detected include, but are not particularly limited to, bacteria, viruses, nucleic acids, proteins, and peptides.

By confirming the presence of a bacterium or virus, it is possible to confirm the presence of a harmful bacterium or virus. By confirming the presence of a substance from a living organism, such as a nucleic acid, protein, or peptide, it becomes possible to detect the presence of a harmful or useful substance in a sample.

In a case in which an in vivo substance is a substance to be detected, it is preferable to perform ultrasonic treatment on a sample in advance to elute or precipitate the substance to be detected in the sample.

Examples of a substance to be detected include, but are not particularly limited to, Escherichia coli (preferably pathogenic Escherichia coli), Streptococcus pneumoniae, Trichophyton, Chlamydia, Staphylococcus aureus, Salmonella enterica, Campylobacter, and Mycobacterium tuberculosis.

Examples of a virus as a substance to be detected include, but are not particularly limited to, influenza virus, norovirus, coronavirus (including new coronavirus), rotavirus, hepatitis virus, varicella-zoster virus, human immunodeficiency virus, and human papillomavirus.

Pathogenic bacteria and viruses are suitable substances to be detected. Examples of pathogenic substances include substances that cause infections or food poisoning, which may be those useful to inspect from the perspective of hygiene control in food manufacturing processes and the like. For example, non-pathogenic E. coli bacteria, which must not be contaminated with food because of the promotion of food spoilage, can be mentioned.

Examples of a nucleic acid, a protein, or a peptide as a substance to be detected include those from bacteria or viruses, and examples other than those from bacteria or viruses include poisonous substances such as snake venom, abnormal prions, and various tumor markers. In addition, a substance that exists in the body and is the target of testing conducted to measure the state of the body, such as a blood test, may be a substance to be detected.

The influenza virus is explained as an example below as a substance to be detected; however, it may be an influenza virus-derived substance such as an influenza virus nucleoprotein or an influenza virus particle. As a material for recognizing the substance to be detected, for example, an antibody that binds to the surface antigen of the influenza virus and an antibody that binds to a nucleoprotein extracted from the influenza virus can be used.

In the present invention, a material for recognizing the substance to be detected which recognizes and binds to a substance to be detected within a sample, is preferably an antibody or an antibody fragment as a material for recognizing the substance to be detected. It may also be a nucleic acid fragment (aptamer) that recognizes a virus such as the influenza virus.

As an antibody or an antibody fragment, an antibody known to recognize a substance to be detected within a sample, which is planned to be detected, or an antibody fragment thereof can be used.

An antibody or an antibody fragment as a material for recognizing the substance to be detected can be produced by a conventionally known method.

An antibody or an antibody fragment is a molecule to which a sugar chain is bound. There is no particular limitation on an antibody fragment as long as it can be labeled with a fluorescent substance or a quencher and recognize a substance to be detected. Examples thereof include, but are not particularly limited to, Fv, Fab, and F(ab′)2.

When a substance to be detected is a nucleic acid, a material for recognizing the substance to be detected may be a nucleic acid having a base sequence complementary to part of the base sequence of the nucleic acid of the substance to be detected. Nucleic acids can be produced by a conventionally known method.

In the present invention, it is preferable to use an antibody or an antibody fragment as a material for recognizing the substance to be detected; however, it is preferable to use the entire antibody molecule containing Fc as it is, and it is preferable that it retains a sugar chain.

Affinity can be maintained by performing fluorescent labeling via the sugar chain in the Fc region using an antibody having a sugar chain.

A polyclonal antibody may be used as an antibody, but a monoclonal antibody is preferable.

A monoclonal antibody can be produced by a conventionally known method. In the present invention, an antibody produced by a known method, or a commercially available antibody can be used.

Since the origin of the antibody is not particularly limited, examples thereof include mammals and may also be experimental animals. Specifically, antibodies derived from mice, rats, rabbits, camels, and the like can be used. The antibody may be a human antibody, a chimeric antibody, a humanized antibody, or the like.

Antibodies have classes such as IgG, IgA, IgM, IgD, and IgE. IgG or IgM may be preferably used, but there is no particular limitation. For example, even when IgG is used, subclasses such as IgG1 to IgG4 are not particularly limited.

The antibody fragment may be a fragment of any of these antibodies described above.

The present invention is advantageous in that a substance to be detected can be detected without performing B/F separation. In particular, in a case in which an antibody or an antibody fragment is used as a material for recognizing the substance to be detected, it is advantageous in that a substance to be detected can be detected without performing B/F separation.

B/F separation used in the present invention refers to the separation of a substance to be detected which is present in free form in water (F), from a substance to be detected which is bound to a material for recognizing the substance to be detected (B). As the most typical example, the ELISA method comprises steps including: 1) immobilizing an antibody to the surface of a container and performing washing; 2) adding a blocking agent for suppressing non-specific adsorption and performing washing; 3) adding a specimen, discarding it after a certain period, performing washing; 4) adding an antibody (enzyme label) that binds to an antigen and performing washing; and 5) adding a specific substrate to detect the enzymatic reaction. The washing operation is involved in each step, which makes each step complicated and the equipment complicated, and the person conducting the test is required to have a certain level of technical proficiency.

In the present invention, a substance to be detected and a material for recognizing the substance to be detected bind to each other in a sample.

Since binding between a substance to be detected and a material for recognizing the substance to be detected takes place in a sample containing the substance to be detected, the sample is preferably a liquid sample.

Binding between a substance to be detected and a material for recognizing the substance to be detected is carried out by mixing a sample as a detection target and the material for recognizing the substance to be detected to confirm the presence of the substance to be detected.

In a case in which a sample with the absence of a substance to be detected is used, binding between a substance to be detected and a material for recognizing the substance to be detected does not take place. However, in the detection method of the present invention, by performing a step of confirming the presence of a substance to be detected within a sample based on changes in the fluorescence intensity measured, it is confirmed that binding between a substance to be detected and a material for recognizing the substance to be detected did not take place.

Therefore, according to the detection method of the present invention, in the step of binding a substance to be detected and a material for recognizing the substance to be detected within a sample, binding takes place in a sample with the presence of a substance to be detected, while on the other hand, binding does not take place in a sample with the absence of a substance to be detected.

In other words, it is understood that the binding step in the detection method of the present invention is substantially a step of mixing a sample and a material for recognizing the substance to be detected.

Binding between a substance to be detected and a material for recognizing the substance to be detected may be binding that results in a reversible equilibrium reaction. It may be the binding of a ligand and a receptor in vivo or the binding in a mode similar to antigen-antibody binding.

The binding step in the present invention is not particularly limited, but the following methods can be mentioned:
(1) a substance to be detected, a material for recognizing the substance to be detected, which is labeled with a fluorescent substance that emits fluorescence at a specific wavelength, and a material for recognizing the substance to be detected, which is labeled with a fluorescent substance that fluoresces at a wavelength different from the specific wavelength or a quencher that absorbs fluorescence with the specific wavelength, are mixed in a sample, thereby binding the substance to be detected and the material for recognizing the substance to be detected in the sample;
(2) a substance to be detected, a material for recognizing the substance to be detected, which is labeled with a fluorescent substance that emits fluorescence at a specific wavelength, and a substance to be detected or a substance other than the substance to be detected, which is recognized at the same site as the substance to be detected, for the material for recognizing the substance to be detected, which is labeled with a fluorescent substance that fluoresces at a wavelength different from the specific wavelength or a quencher that absorbs fluorescence with the specific wavelength, are mixed in a sample, thereby binding the substance to be detected and the material for recognizing the substance to be detected in the sample; and
(3) a substance to be detected and a material for recognizing the substance to be detected, which is labeled with a fluorescent substance that emits fluorescence at a specific wavelength, are mixed in a sample, thereby binding the substance to be detected and the material for recognizing the substance to be detected.

In a case in which the binding step is (1) above, it is preferable that the recognition site for a substance to be detected differs between a material for recognizing the substance to be detected, which is labeled with a fluorescent substance that emits fluorescence at a specific wavelength, and a material for recognizing the substance to be detected, which is labeled with a fluorescent substance that fluoresces at a wavelength different from the specific wavelength or a quencher that absorbs fluorescence with the specific wavelength. As the recognition site is different, two kinds of material for recognizing the substance to be detected can simultaneously bind to a substance to be detected, resulting in a so-called sandwich structure.

In this case, the order of mixing a material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength with a substance to be detected within a sample, is not particularly limited. However, it is preferable to mix and bind a material for recognizing the substance to be detected and a substance to be detected by adding a sample which needs to be confirmed whether to contain a substance to be detected in a space with the presence of a material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength, or vice versa. It is also possible to mix a substance to be detected within a sample with one of materials for recognizing the substance to be detected in advance and then with the other material for recognizing the substance to be detected. In this case, the timing of mixing the other material for recognizing the substance to be detected may be after the concentration of a binding product. In addition, after a substance to be detected or a material for recognizing the substance to be detected is concentrated, a binding product may be formed. The concentration and formation of a binding product may be performed at the same time.

In a case in which the binding step is (1) above, a material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength, bind to a substance to be detected. Therefore, the substance to be detected is schematically sandwiched between two different materials for recognizing the substance to be detected.

In a case in which the binding step is (2) above, a substance to be detected which needs to be confirmed whether to be present in a sample, is selected as a substance to be detected for a substance to be detected which is labeled with a fluorescent substance that fluoresces at a wavelength different from the specific wavelength or a quencher that absorbs fluorescence with the specific wavelength. In addition, a substance other than a substance to be detected which is recognized at the same site as the substance to be detected, may be used for a material for recognizing the substance to be detected which is labeled with a fluorescent substance that fluoresces at a wavelength different from the specific wavelength or a quencher that absorbs fluorescence with the specific wavelength. A substance other than the substance to be detected is labeled with a fluorescent substance that fluoresces at a wavelength different from the specific wavelength or a quencher that absorbs fluorescence with the specific wavelength and is recognized at the same site as the substance to be detected in the material for recognizing the substance to be detected. Therefore, it behaves in a way similar to that of the substance to be detected. Hereinafter, the description of a substance to be detected which is labeled with a fluorescent substance that fluoresces at a wavelength different from the specific wavelength or a quencher that absorbs fluorescence with the specific wavelength used herein, also applies to a substance other than the substance to be detected which is recognized at the same site as the substance to be detected, for a material for recognizing the substance to be detected. A substance other than the substance to be detected which is recognized at the same site as the substance to be detected, for a material for recognizing the substance to be detected may be a substance understood as a pseud-substance to be detected.

In this case, it is also possible to mix a substance to be detected with a material for recognizing the substance to be detected in advance and then with another substance to be detected. In this case, the timing of mixing another substance to be detected may be after the concentration of a binding product. In addition, after a substance to be detected or a material for recognizing the substance to be detected is concentrated, a binding product may be formed. The concentration and formation of a binding product may be performed at the same time.

In a case in which the binding step is (2) above, a substance to be detected and a substance to be detected labeled with a fluorescent substance or a quencher in a sample bind competitively to a material for recognizing the substance to be detected.

It is preferable to bind a material for recognizing the substance to be detected, which is labeled with a fluorescent substance that emits fluorescence at a specific wavelength, and a material for recognizing the substance to be detected, which is labeled with a fluorescent substance that fluoresces at a wavelength different from the specific wavelength or a quencher that absorbs fluorescence with the specific wavelength and then mix the binding product with a substance to be detected within a sample. It is also possible to bind a material for recognizing the substance to be detected, which is labeled with a fluorescent substance that emits fluorescence at a specific wavelength, and a substance to be detected within a sample and then mix the binding product with a material for recognizing the substance to be detected, which is labeled with a fluorescent substance that fluoresces at a wavelength different from the specific wavelength or a quencher that absorbs fluorescence with the specific wavelength.

A fluorescent substance or a quencher needs to be bound at a site where the binding between a substance to be detected and a material for recognizing the substance to be detected is not inhibited or least inhibited as possible. In addition, in a case where two different fluorescent substances are used, when a fluorescent substance and a quencher are used, it is preferable that a site for binding to a substance to be detected or a material for recognizing the substance to be detected is selected such that the fluorescent substance and the quencher are present in the vicinity in a binding product, specifically, they can give and receive optical excitation energy to each other.

In the present invention, if appropriate, a fluorescent substance for labeling a substance to be detected or a material for recognizing the substance to be detected may be selected.

As the fluorescent substance, a substance that satisfies some or all of the following requirements is preferably used.
The excitation wavelength is long, preferably 350 nm or more, and more preferably 400 nm or more.
The fluorescence wavelength is short, preferably 1500 nm or less.
The extinction coefficient is high, preferably, the molar extinction coefficient is 10,000 or more.
The quantum yield is high, preferably 0.1 or more.
In a case in which a plurality of fluorescent substances are used, at a wavelength for exciting one fluorescent substance, another fluorescent substance is not excited, and when the fluorescence of another fluorescent substance is measured, the fluorescence of the one fluorescent substance does not overlap. In this case, to allow the fluorescence of one fluorescent substance to promote the excitation of another fluorescent substance efficiently, it is preferable that the peak of the fluorescence spectrum of the one fluorescent substance is sufficiently close to the peak of the excitation spectrum of another fluorescent substance. Further, it is preferable that the fluorescence spectrum of one fluorescent substance is sufficiently narrow, and the excitation spectrum of another fluorescent substance is sufficiently broad. It is more preferable that the difference between the positions of the excitation spectrum peak and the fluorescence spectrum peak (Stokes shift) is sufficiently large.

Further, it is preferable that fluorescent substances satisfy some or all of the following requirements.
The water solubility of a substance to be labeled is not inhibited.
The specific binding between a substance to be detected and a material for recognizing the substance to be detected is not inhibited after labeling.
Labeling does not cause aggregation of unlabeled substance and/or enhancement of light scattering.

In the present invention, if appropriate, a quencher for labeling a substance to be detected or a material for recognizing the substance to be detected may be selected.

The quencher must be a substance that satisfies the following requirements in addition to the requirements described above for the fluorescent substance.
Even with energy transfer, there is no secondary fluorescence, or fluorescence is emitted at a wavelength further away from the measurement range.

In the detection method of the present invention, at least one of fluorescent substances is preferably a quantum dot.

For fluorescent labeling, fluorescent particles made of a semiconductor having a diameter of several nanometers (nm), commonly called quantum dots, have an extinction coefficient and quantum yield several tens of times higher than organic dyes. Their fluorescence is strong, and half-width is narrow, making it easy to obtain high performance as a system. The narrow half-width allows excitation energy to be received by the other dye (acceptor) without leaking within the emission wavelength range during energy transfer, which is called the FRET phenomenon. Another great advantage is that by using a method of binding a quantum dot to a sugar chain of an antibody, it is possible to completely avoid the reduction in affinity due to chemical manipulation of the Fv region, which may occur in some cases.

According to the present invention, it is not necessary to be a FRET-like energy transfer in the precise sense; however, it is sufficient that an energy transfer, which results in a change that can be distinguished advantageously, occurs.

In the present invention, a substance to be detected, a substance other than the substance to be detected which is recognized at the same site as the substance to be detected for the substance to be detected and a material for recognizing the substance to be detected, or a material for recognizing the substance to be detected, may be physically or chemically bound to a carrier particle.

Whether to use a material for recognizing the substance to be detected which is bound to a carrier particle, may be determined based on the nature of the substance to be detected.

In the detection method of the present invention, a substance to be detected, a material for recognizing the substance to be detected, or a binding product of a substance to be detected and a material for recognizing the substance to be detected is locally concentrated by electrophoresis or dielectrophoresis. Therefore, the formation of a binding product of a substance to be detected and a material for recognizing the substance to be detected may be a pre-step or a post-step of the concentration step. Concentration and binding may be carried out simultaneously. For example, when mixing a substance to be detected and a material for recognizing the substance to be detected, as long as electrophoresis or dielectrophoresis is performed, it can be said that the concentration step and the binding step are carried out simultaneously.

For example, a material for recognizing the substance to be detected which is not bound to a carrier particle, can be used for a binding product of a substance to be detected and a material for recognizing the substance to be detected which can be locally concentrated by electrophoresis or dielectrophoresis, although not particularly limited thereto. Even for a binding product of a substance to be detected and a material for recognizing the substance to be detected, which can be locally concentrated by electrophoresis or dielectrophoresis, it is also possible to bind a material for recognizing the substance to be detected to a carrier particle so as to carry out electrophoresis or dielectrophoresis more efficiently.

Therefore, whether to allow a carrier particle to physically or chemically bind to a material for recognizing the substance to be detected can be determined by confirming whether it is possible to locally concentrate a substance to be detected per se, a material for recognizing the substance to be detected per se, or a binding product of a substance to be detected and a material for recognizing the substance to be detected by electrophoresis or dielectrophoresis before carrying out the detection method of the present invention.

In a case in which it is impossible to locally concentrate a substance to be detected per se, a material for recognizing the substance to be detected per se, or a binding product of a substance to be detected and a material for recognizing the substance to be detected by electrophoresis or dielectrophoresis, a carrier particle may physically or chemically bind to a substance to be detected per se, a material for recognizing the substance to be detected per se, or a binding product of a substance to be detected and a material for recognizing the substance to be detected.

A carrier particle may be selected based on the efficiency of local concentration by electrophoresis or dielectrophoresis.

A method for allowing a material for recognizing the substance to be detected to physically or chemically bind to a carrier particle is not particularly limited. Binding may be carried out by a conventionally known method or the manual attached to the carrier particle.

In the present invention, a material for recognizing the substance to be detected, which is physically or chemically bound to a carrier particle, may be contained.

The material for recognizing the substance to be detected may or may not be labeled with a fluorescent substance or a quencher or may be an unlabeled material for recognizing the substance to be detected.

In this case, for example,

The material for recognizing the substance to be detected which is labeled with a fluorescent substance that emits a specific wavelength, and a material for recognizing the substance to be detected which is labeled with a fluorescent substance that fluoresces at a wavelength different from the specific wavelength or a quencher that absorbs fluorescence with the specific wavelength simultaneously, bind to the substance to be detected in the sample, and
a material for recognizing the substance to be detected which is physically or chemically bound to the carrier particle, is further contained, and the substance to be detected and the material for recognizing the substance to be detected form a binding product in the sample.

### Alternatively,

the substance to be detected, the material for recognizing the substance to be detected which is labeled with a fluorescent substance that emits fluorescence at a specific wavelength, a substance to be detected or a substance other than the substance to be detected which is recognized at the same site as the substance to be detected, for the material for recognizing the substance to be detected, which is labeled with a fluorescent substance that fluoresces at a wavelength different from the specific wavelength or a quencher that absorbs fluorescence with the specific wavelength, and a material for recognizing the substance to be detected which is physically or chemically bound to a carrier particle, may be used in the sample.

Further, a labeled material for recognizing the substance to be detected may be physically or chemically bound to a carrier particle without using an unlabeled material for recognizing the substance to be detected, which is physically or chemically bound to a carrier particle.

The carrier particle used in the detection method of the present invention is not particularly limited as long as a binding product with a substance to be detected can be locally concentrated when the carrier particle is allowed to physically or chemically bind to a material for recognizing the substance to be detected. Conventionally known carrier particles can be used. In addition, the carrier particle used in the Examples is an example of a carrier particle that can be preferably used in the present invention.

Specific examples of a carrier particle include, but are not particularly limited to, a metal fine particle, a metal oxide fine particle, a non-metal fine particle, a metal-coated resin fine particle, and a non-infectious spherical biological fine particle.

Specific examples of a metal fine particle include gold, silver, platinum, titanium, palladium, iron, and aluminum.

Specific examples of a metal oxide fine particle include titanium dioxide, aluminum oxide, magnesium oxide, indium-tin oxide (ITO), and antimony-tin oxide (ATO).

Specific examples of a non-metal fine particle include fine particles made of magnetic particles, graphite, polystyrene, and conductive resin.

A non-infectious spherical biological fine particle may be any of those belonging to bacteria, oomycetes, oomycetes, fungi, and the like. Specific examples thereof include Lactococcus and Saccharomyces cerevisiae (yeast).

Fine particles whose surfaces are plated with conductive metal, sold under the trade names of Micropearl and Micropearl Au manufactured by SEKISUI CHEMICAL CO., LTD., may also be used.

The particle size of the carrier particles is not particularly limited and is appropriately selected depending on the carrier particle used.

Although the particle size is not particularly limited, for example, the particle size of colloidal gold particles is from 30 to 100 nm and preferably from 40 to 60 nm, the particle size of polystyrene fine particles is preferably in a range of from 100 nm to 3 μm, and the particle size of Lactococcus and yeast is preferably in a range of from 1 to 5 μm.

A fine particle having a particle size of 5 μm or less can be preferably used as a metal-coated resin fine particle.

In the present invention, it is also possible to bind a substance to be detected and a material for recognizing the substance to be detected within a sample so as to form a binding product of the substance to be detected and the material for recognizing the substance to be detected and then measure changes in the fluorescence intensity while locally concentrating the binding product by electrophoresis or dielectrophoresis.

Here, the material for recognizing the substance to be detected may be bound to a carrier particle.

Electrophoresis or dielectrophoresis of the binding product can be carried out by a conventionally known method.

In the present invention, dielectrophoresis is preferably used. Meanwhile, it is possible to perform dielectrophoresis to concentrate a binding product of a substance to be detected and a material for recognizing the substance to be detected so as to detect a desired substance to be detected. Also, in a case in which the binding product is concentrated by electrophoresis, as in the case of dielectrophoresis, a desired substance to be detected can be detected.

One method for applying an electric field to a binding product for performing electrophoresis or dielectrophoresis may be applying a direct current. Fine particles in water are generally charged with either a positive or negative surface charge. For example, E. coli is negatively charged. When a gel electrode is used to apply a DC voltage to a sample containing floating E. coli, the E. coli cells aggregate at the positive electrode as if forming a solid near the gel. The present inventors previously confirmed that by cutting off the voltage at this time, this solidified state will be loosened, and once the polarity is reversed, the cells will move in the opposite direction. Measuring E. coli in this state increases the sensitivity of the detection system.

Applying a direct current (electrophoresis) may induce electrolysis of water. The same principle may cause the electrode material to be ionized and dissolved. Thus, it is preferable to apply an alternating current (dielectrophoresis).

When applying an alternating current, it is known that electrolysis does not occur even when applying the current up to several volts through a metal electrode, and particles move between regions with different electric field densities due to a phenomenon called dielectrophoresis. At this time, it is known that the particles are not affected by surface charge but by the dielectricity and conductivity of the particles and solvent and the frequency of the electric field (R. Pethig, BIOMICROFLUIDICS, 4, 022811(2010)).

The voltage to be applied may be, for example, from 0.1 to 10 V, from 1 to 5 V, from 1 to 4 V, or from 2 to 4 V, although not particularly limited thereto. The frequency is, for example, from 100 Hz to 200 MHz, preferably in the MHz order or around 2 MHz, although not particularly limited thereto.

In dielectrophoresis, when an alternating current is used, the governing factors of dielectrophoresis that occur will vary depending on the permittivity and conductivity of the target particles and solvent, the applied voltage, and the frequency (in the case of a sine wave). Positive dielectrophoresis is explained above, which is the case in which particles gather in an area where the electric field density is high, such as a corner of an electrode. In some cases, negative dielectrophoresis may occur, and particles may move away from the electrode depending on the combination of frequency band, permittivity, and conductivity.

In the present invention, dielectrophoresis may be positive or negative dielectrophoresis; however, positive dielectrophoresis is preferable.

In the present invention, changes in the fluorescence intensity may be measured while locally concentrating a binding product. In this case, it is possible to confirm the presence of a substance to be detected within a sample without B/F separation.

Measuring changes in the fluorescence intensity during concentration in the present invention means that B/F separation is not performed. As B/F separation is not performed, the fluorescence intensity may be measured after locally concentrating a binding product.

In addition, in the present invention,
a step of locally concentrating a substance to be detected, a material for recognizing the substance to be detected, or a binding product of a substance to be detected and a material for recognizing the substance to be detected within a sample by electrophoresis or dielectrophoresis,
a step of optionally binding the substance to be detected and the material for recognizing the substance to be detected in the sample, and
a step of measuring changes in fluorescence intensity of a binding product of the substance to be detected and the material for recognizing the substance to be detected are included. Concentration and fluorescence intensity measurement may be performed simultaneously, concentration and binding may be performed simultaneously, binding and fluorescence intensity measurement may be performed simultaneously, or concentration, binding, and fluorescence intensity measurement may be performed simultaneously.

In the present invention, to improve the degree of freedom in the design, based on an idea to separate concentration and fluorescence intensity measurement, a step of performing detection from changes in fluorescence using a system in which the fluorescence intensity varies and a step of concentrating a reaction system independently from the system by electrophoresis or dielectrophoresis to improve the sensitivity of the entire system are separately and independently performed in a more preferred embodiment.

Further, in one preferred embodiment,
a substance to be detected, a material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength, are mixed in a sample, thereby binding the substance to be detected and the material for recognizing the substance to be detected.

Alternatively, a substance to be detected, a material for recognizing the substance to be detected which is labeled with a fluorescent substance that emits fluorescence at a specific wavelength, and a substance to be detected or a substance other than the substance to be detected which is recognized at the same site as the substance to be detected, for the material for recognizing the substance to be detected, which is labeled with a fluorescent substance that fluoresces at a wavelength different from the specific wavelength or a quencher that absorbs fluorescence with the specific wavelength, are mixed in a sample, thereby binding the substance to be detected and the material for recognizing the substance to be detected in the sample.

Alternatively, the material for recognizing the substance to be detected which is labeled with a fluorescent substance that emits a specific wavelength, and a material for recognizing the substance to be detected which is labeled with a fluorescent substance that fluoresces at a wavelength different from the specific wavelength or a quencher that absorbs fluorescence with the specific wavelength simultaneously, bind to the substance to be detected in the sample, and
a material for recognizing the substance to be detected, which is physically or chemically bound to the carrier particle, is further contained, and the substance to be detected and the material for recognizing the substance to be detected, form a binding product in the sample.

Furthermore, in another preferred embodiment,
a material for recognizing a substance to be detected, which is physically or chemically bound to a carrier particle, is allowed to or not to bind to the substance to be detected, locally concentrated by electrophoresis or dielectrophoresis, and optionally the substance to be detected and the material for recognizing the substance to be detected are bound to each other in a sample. Changes in the fluorescence intensity are measured for the substance to be detected by any of the methods described above as a preferred embodiment.

If appropriate, fluorescence intensity measurement can be set depending on the fluorescent substance.

Conditions for fluorescence intensity measurement can also be set as appropriate. The excitation light generally has a wavelength of from 300 to 600 nm, preferably from 350 nm or more, and more preferably 400 nm or more.

For fluorescence intensity measurement, it is possible to perform measurement using a filter+an optical sensor (photodiode, phototransistor) or to take an image of the illuminated state with a smartphone image sensor and compare the image with a known electrode shape for image recognition, thereby extracting only the illuminated part which makes it possible to enhance contrast and perform more accurate detection.

The step of measuring changes in the fluorescence intensity in the present invention is not particularly limited, but the following methods can be mentioned:

In a case in which the binding step is (1) or (2) above, and two different fluorescent substances are used, the fluorescent substance that emits fluorescence at a specific wavelength is a fluorescent substance that is excited at wavelength 1 and emits wavelength 2, the fluorescent substance that fluoresces at a wavelength different from the specific wavelength is a fluorescent substance that absorbs wavelength 2 and emits wavelength 3, and excitation is induced at wavelength 1 so as to measure the fluorescence intensity of wavelength 3 or the fluorescence intensity of wavelengths 2 and 3.

In a case in which the binding step is (1) or (2) above, and a quencher is used, the fluorescent substance that emits fluorescence at a specific wavelength is a fluorescent substance that is excited at wavelength 1 and emits wavelength 2, the quencher that absorbs fluorescence with the specific wavelength is a fluorescent substance that absorbs wavelength 2 but does not emit fluorescent at least in a measurement wavelength range, and excitation is induced at wavelength 1 so as to measure a fluorescence intensity of wavelength 2.

In a case in which the binding step is (3) above, the fluorescent substance that emits fluorescence at a specific wavelength is a fluorescent substance that is excited at wavelength 1 and emits wavelength 2, and excitation is induced at wavelength 1 so as to measure a fluorescence intensity of wavelength 2.

In the measurement of changes in the fluorescence intensity, measuring changes in the fluorescence wavelength or changes in the degree of fluorescence may be regarded as a step of measuring changes in fluorescence intensity of a binding product of the substance to be detected and the material for recognizing the substance to be detected.

In a case in which the binding step is (1) above, and two different fluorescent substances are used, it corresponds to a fluorescence intensity measurement method utilizing energy transfer due to the presence of two different fluorescent substances in the vicinity of the substance to be detected via the material for recognizing the substance to be detected, by which high sensitivity can be achieved by observing energy transfer between the two different fluorescent substances. It is particularly preferable to measure the fluorescence emitted from a fluorescent substance that fluoresces at a wavelength different from the specific wavelength, which was not observed before mixing.

In a case in which the binding step is (1) above, and a fluorescent substance and a quencher are used, it corresponds to a fluorescence intensity measurement method utilizing energy transfer due to the presence of a fluorescent substance and a quencher in the vicinity of the substance to be detected via the material for recognizing the substance to be detected, by which high sensitivity can be achieved by observing energy transfer between the fluorescent substance and the quencher. It is particularly preferable to measure the fluorescence emitted from a fluorescent substance that fluoresces at the specific wavelength, which is reduced by mixing.

In a case in which the binding step is (1) above, and a carrier particle is used, either a material for recognizing the substance to be detected which is labeled with a fluorescent substance that emits fluorescence at a specific wavelength, or a material for recognizing the substance to be detected which is labeled with a fluorescent substance that fluoresces at a wavelength different from the specific wavelength or a quencher may be carried by a carrier particle; however, it is preferable that a material for recognizing the substance to be detected which is labeled with a fluorescent substance that emits fluorescence at a specific wavelength is carried by a carrier particle.

In a case in which the binding step is (2) above, and two different fluorescent substances are used, it corresponds to a fluorescence intensity measurement method utilizing energy transfer due to the presence of two different fluorescent substances in the vicinity, by which high sensitivity can be achieved by observing energy transfer between the two different fluorescent substances. It is particularly preferable to measure the fluorescence emitted from a fluorescent substance that fluoresces at a wavelength different from the specific wavelength, which is reduced by mixing. In this case, as the substance to be detected replaces the substance to be detected labeled with a fluorescent substance to bind to the material for recognizing the substance to be detected in the sample, measurement utilizing the phenomenon caused by the release of the substance to be detected labeled with a fluorescent substance is preferred.

In a case in which the binding step is (2) above, and a fluorescent substance and a quencher are used, it corresponds to a fluorescence intensity measurement method utilizing energy transfer due to the presence of a fluorescent substance and a quencher in the vicinity, by which high sensitivity can be achieved by observing energy transfer between the fluorescent substance and the quencher. It is particularly preferable to measure the fluorescence emitted from a fluorescent substance that fluoresces at the specific wavelength, which is increased by mixing. In this case, as the substance to be detected replaces the substance to be detected labeled with a fluorescent substance to bind to the material for recognizing the substance to be detected in the sample, measurement utilizing the phenomenon caused by the release of the substance to be detected labeled with a quencher is preferred.

In a case in which the binding step is (2) above, a material for recognizing the substance to be detected which is labeled with a fluorescent substance that emits fluorescence at a specific wavelength, and a substance to be detected which is labeled with a fluorescent substance that fluoresces at a wavelength different from the specific wavelength or a quencher, are used, however, the substance to be detected may be labeled with a fluorescent substance that fluoresces at a specific wavelength, and the material for recognizing the substance to be detected may be labeled with a fluorescent substance or a quencher that fluoresces at a wavelength different from the specific wavelength.

In a case in which the binding step is (3) above, a material for recognizing the substance to be detected is present throughout a sample, and fluorescence from a fluorescent substance labeled on the material for recognizing the substance to be detected is observed as the background. In a case in which the binding step is (1) or (2) above, a material for recognizing the substance to be detected, which is labeled with a fluorescent substance that fluoresces at a specific wavelength may be carried by a carrier particle. In a case in which the binding step is (3) above, it is preferable that a substance to be detected per se is concentrated by electrophoresis or dielectrophoresis.

In a case in which the binding step is any one of (1) to (3) above, in each of the embodiments described above as the fluorescence intensity measurement step in the present invention, a material for recognizing the substance to be detected which is physically or chemically bound to the carrier particle, may be further contained, and the substance to be detected and the material for recognizing the substance to be detected may form a binding product in the sample. In this case, with the presence of the material for recognizing the substance to be detected which is physically or chemically bound to the carrier particle, the substance to be detected is locally concentrated via binding with the material for recognizing the substance to be detected. In addition, the material for recognizing the substance to be detected is physically or chemically bound to a carrier particle but not labeled with a fluorescent substance or a quencher.

In a case in which a carrier particle physically or chemically binds to a substance to be detected or a material for recognizing the substance to be detected, as described herein, the carrier particle is preferably a particle that promotes dielectrophoresis.

Any of a fluorescent substance that fluoresces at a specific wavelength, a fluorescent substance that fluoresces at a wavelength different from the specific wavelength, or a quencher used in the present invention may be a carrier particle.

In the detection method of the present invention, highly sensitive measurements can be performed without requiring specialized equipment, environment, knowledge, and/or technology. Therefore, the present invention enables testing for infectious diseases using conventionally impossible samples, on-site testing where conventional testing was necessary but could not be performed, and testing for items that could not be tested in principle with genetic testing.

More specifically, in the case of detecting the influenza virus, it becomes possible to use saliva instead of nasal secretion as a sample, to use waterfront areas such as customs offices as inspection sites, and to use sites such as food processing factories and distribution processes that previously required culturing for several days directly as testing sites, making it possible to obtain results on-site. Alternatively, pathogenic factors that do not include genes, such as abnormal prions, can also be tested.

A detection device for a substance to be detected within a sample for carrying out the detection method of the present invention is exemplified below or in the Examples, which do not restrict the present invention.

The detection device comprises: a detection cell having a pair of microelectrodes in a liquid reservoir capable of introducing and holding an aqueous sample, the microelectrodes being electrically connected to an external or internal voltage generator; means of concentrating a substance to be detected within a sample by electrophoresis or dielectrophoresis by applying a direct current or alternating current between the electrodes while being in contact with the sample; and detection means capable of specifically binding to a substance to be detected and having at least a material for recognizing the substance to be detected, which is labeled with fluorescence in advance, and measuring the fluorescence intensity of the label, and the detection device allows the aqueous sample to move and evaluates the fluorescence intensity without performing B/F separation between a binding product of a substance to be detected and a material for recognizing the substance to be detected (B) and a material for recognizing the substance to be detected in free form (F), thereby qualitatively or quantitatively measuring the state of binding between the substance to be detected and the material for recognizing the substance to be detected, making it possible to measure the presence of the substance to be detected in the sample.

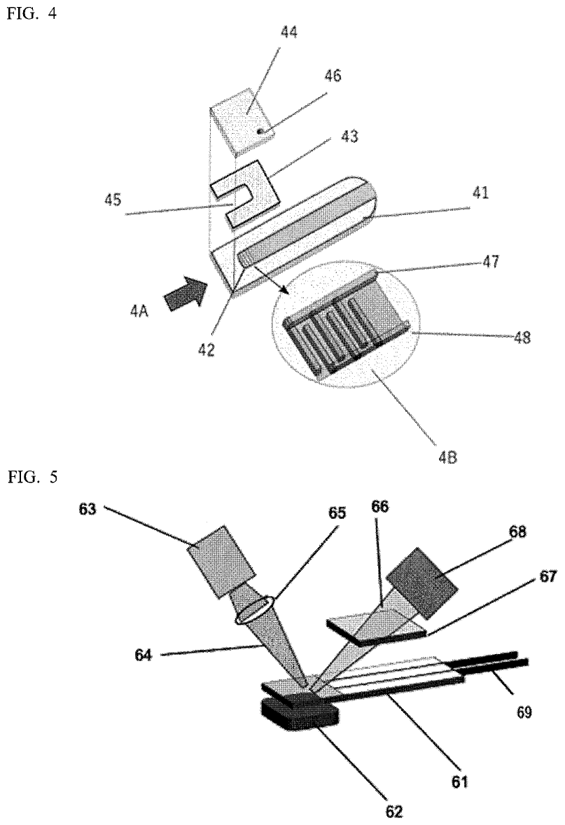

FIG. 4 shows a typical configuration diagram of a detection cell. FIG. 5 shows a typical configuration diagram of a detection device.

The detection device comprises a light source 63 and a lens 65 as detection means, and an excitation light 64 with a wavelength that excites a fluorescent substance is irradiated on a detection cell 61 from the light source 63 via the lens 65. To confirm the presence of a substance to be detected, the detection device further comprises a light sensor 68 and a light filter 67 for measuring the fluorescence intensity. Fluorescence 66 of a detection wavelength or fluorescence 66 other than the fluorescence of a detection wavelength is collected by the light filter 67 such that the light sensor 68 measures the fluorescence intensity. Preferably, the light filter 67 cuts fluorescence with a wavelength other than the detection wavelength. The detection cell comprises means of concentrating a substance to be detected within a sample by electrophoresis or dielectrophoresis, by which a microelectrode 42 is printed on a substrate 41. A pair of microelectrodes are shown as an electrode 1 (47) and an electrode 2 (48). A liquid reservoir formed in the detection device is defined by substrate 41, spacer 43, and cover 44 and is shown as capillary 45. An air hole 46 is formed in the cover 44. In FIG. 4, 4A denotes the direction of sample introduction.

A terminal 69 is connected as concentration means to the electrodes 1 (47) and 2 (48). For example, when performing dielectrophoresis, the terminal 69 applies a high-frequency wave with optimal voltage and frequency from a function generator (not shown).

The detection device may comprise means of forcibly stirring a sample by applying vibration to the cell after introducing the sample into the detection cell. For example, when a substance to be detected is a virus nucleoprotein, the means is used to destroy the virus envelope to expose the nucleoprotein during detection. In FIG. 5, the means is exemplified as vibrator 62.

In addition, upon fluorescence intensity measurement by the detection means, in the light sensor 68, fluorescence is photographed with an image sensor through a light filter 67 that does not pass excitation light, electronic means of image processing that refers to the shape of the microelectrode and extracts it is provided, the unextracted part is regarded as background noise, the information on changes in fluorescence intensity is corrected, and the contrast is enhanced, such that more sensitive measurements become possible.

The detection device may further comprise a crusher capable of processing a sample containing a substance to be detected into a slurry close enough to a liquid that can be dispersed in water and introduced into a detection cell as a sample in a detection method. In this case, a solid containing a substance to be detected can also be introduced as a sample.

Appropriate amounts of a substance to be detected or a material for recognizing the substance to be detected labeled with a fluorescent substance or quencher, a pH adjustment buffer ideal for reactions, a surfactant, and the like may adhere to the detection cell provided in the detection device of the present invention. These may be added to the detection cell as a solution in advance and lyophilized to adhere to the detection cell wall.

A reaction solution used for fluorescence detection in the present invention is preferably a solution in which a binding reaction between a substance to be detected and a material for recognizing the substance to be detected, electrophoresis, or dielectrophoresis can be carried out. For example, a sample containing a substance to be detected may be mixed in the solution to detect the substance to be detected. For example, the solution described in the Examples can be favorably used as the reaction solution. Desirably, a solvent that minimizes conductivity in a range that does not inhibit a reaction between a substance to be detected and a material for recognizing the substance to be detected, for example, non-ionized water or a mixture of this and sugar alcohol may be used. In addition, in consideration of the fact that a substance to be detected and a material for recognizing the substance to be detected can be bound and concentrated using the same solvent, for example, a phosphate buffer may be used, and PBS at a concentration of 1 mM or less may be used.

The pH adjustment buffer used herein is not particularly limited. It may be selected appropriately considering a substance to be detected, which is planned to be detected, and a binding reaction between a substance to be detected and a material for recognizing the substance to be detected.

A surfactant is used, for example, to degrade a virus and extract the internal nuclear protein, and is also used to guide a sample into the cell by capillary action. One type of surfactant may be used, or two or more types may be used depending on the required effects. For example, TritonX-100 can be appropriately used to extract the nucleoprotein from the influenza virus.

In the present invention, a small tube is prepared separately from the detection cell, and the components used for detection may be mixed in the tube and then introduced into the detection cell. In this case, the tube may contain a surfactant and other components in advance. The method of introduction into the tube is not particularly limited, but a surfactant or the like may be present in the tube by freeze-drying. Alternatively, the surfactant and other components may be contained separately in separate containers and mixed with the sample in the tube before being placed in the cell.

In the present invention, calibration may be performed by measuring with or without a sample, detection may be performed using a standard solution, detection may be performed using a standard reference value for each lot, and quality control may be performed thoroughly such that it is possible to perform detection without using reference values.

### EXAMPLES

The present invention will be further explained below with reference to Examples, but the present invention is not limited to the following Examples.

### Example 1

### Labeling of Antibody

FIA3298 (BIO MATRIX RESEARCH, INC.) was used as an antibody that binds to the nucleoprotein of influenza A virus. Quantum dots (Qdot565, Thermo Fisher) were chemically labeled on sugar chains in the Fc region of the antibody according to the method of the Qdot Antibody Conjugation Kit (BIO MATRIX RESEARCH, INC.). The fluorescence spectrum of the antibody Ab1Qd565 chemically labeled with quantum dots was measured using a fluorescence spectrophotometer (F2500, Hitachi High-Tech Corporation) and had a fluorescence peak at 561 nm (FIG. 1).

As an example, an antibody labeled with quantum dots is represented by the following formula.

### Labeling of Nucleoprotein (NP)

AAM75159.1 (Sino Biological) was used as an influenza A (H1N1) nucleoprotein (NP) created by genetic recombination. In addition, the NHS ester of QSY9 (Thermo Fisher) was used as a quencher.

The NHS ester of QSY9 is represented by the following formula.

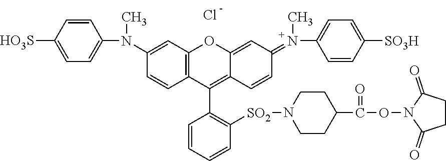

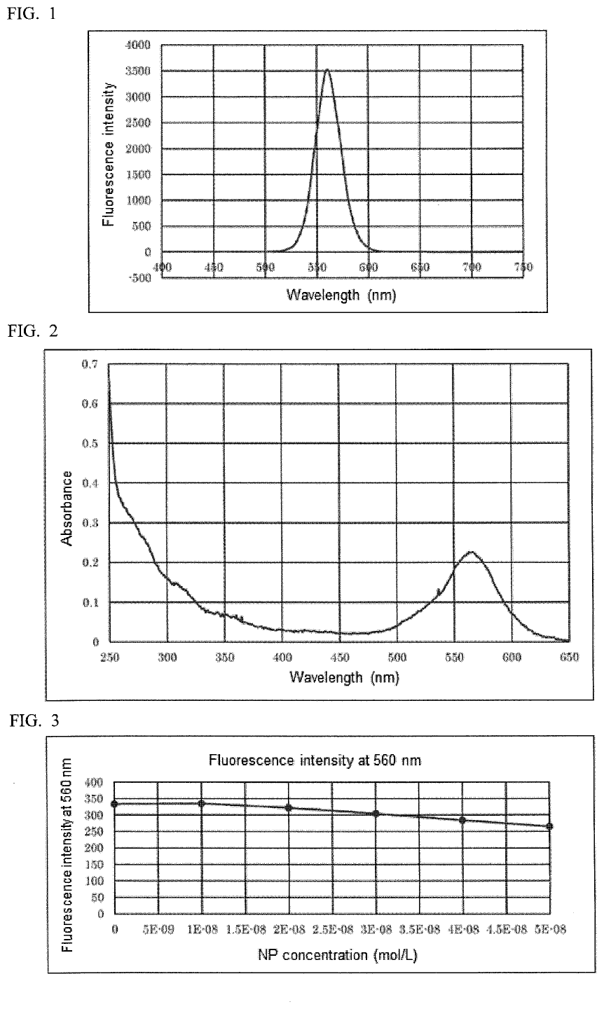

The NHS ester of QSY9 was dissolved in dimethyl sulfoxide (DMSO). The NHS ester DMSO solution of QSY9 was added to an aqueous solution of NPs dissolved in 10 mM sodium bicarbonate buffer (pH 8.0) such that the number of QSY9 molecules was 30 times the number of NP molecules and left at room temperature overnight. The quencher-labeled nucleoprotein NPQSY9 was purified from the mixed solution using a Micro Biospin Column 30 manufactured by Biorad. Purification conditions followed the protocol provided by Biorad, except that the developing solution was PBS (pH 7.2). FIG. 2 shows the absorption spectrum of the fraction obtained by purification, measured using a UV/VIS spectrophotometer (UV2500PC, SHIMADZU CORPORATION).

A comparison between FIGS. 1 and 2 shows that the fluorescence peak of the antibody Ab1Qd565 chemically labeled with quantum dots and the absorption peak of the quencher in the nucleoprotein NPQSY9 labeled with the quencher overlapped almost ideally.

### Confirmation Experiment on Quenching

Ab1Qd565 was diluted with PBS to a concentration of 10 nM. Fluorescence spectra of 200 μL of the diluted solution were measured using a fluorescence spectrophotometer (F2500, Hitachi, Ltd.) using a microcell. This solution was mixed with 2 μL of NPQSY9 adjusted to a protein concentration of 10 μM for each measurement, and changes in the fluorescence intensity at 561 nm were observed (FIG. 3).

The results shown in FIG. 3 confirmed that the fluorescence with a peak at 561 nm was quenched due to the binding of NPQSY9 and Ab1Qd565 in the solution.

### Dielectrophoresis of Polystyrene Fine Particles

An aqueous dispersion of polystyrene fine particles having a particle size of 3 μm (Polysciences) was diluted 100,000 times with non-ionized water. The conductivity was measured using a conductivity meter (EC-33, HORIBA, Ltd.) and found to be 13 μS/cm.

Subsequently, dielectrophoresis of the polystyrene fine particles was observed under the following conditions.
Cell: Cell in which a cover glass was attached to the microelectrode part of a comb-shaped microelectrode (Au; distance between electrodes: 5 μm) manufactured by BAS via a 12-μm spacer.
High-frequency generator: High-frequency generator: AFG3022 function generator manufactured by TEKTRONIX, INC.
Oscilloscope: 54602B manufactured by Hewlett-Packard
Microscope: VHX900 with 1000× lens manufactured by TEKTRONIX, INC.

About 10 μL of the diluted solution of polystyrene fine particles prepared as described above was placed on the microelectrode, and sweep voltage and frequency were applied during microscopic observation. It was confirmed that polystyrene fine particles lined up along the contour of the electrode under the condition of 2 V/1 kHz.

In other words, positive dielectrophoresis in polystyrene fine particles was observed. The time required for the fine particles to aggregate and reach a steady state was about from 1 to 2 seconds. In addition, positive dielectrophoresis was similarly observed when yeast was used, and yeast was concentrated along the electrode.

### Carrying of Antibody

Ab1Qd565 was carried on the surfaces of polystyrene fine particles using the method specified by the latex particle manufacturer. After that, blocking treatment was performed with BSA, thereby preventing unnecessary non-specific adsorption. NPQSY9 was mixed with this such that the number of antibody molecules and the number of NP molecules were equal, and quenching was caused in advance.

Using DFC360FX manufactured by Leica Microsystems as a fluorescence microscope, dielectrophoresis was performed using the above-described cell under observation with an ebq100 light source, and it was confirmed that the fluorescence derived from Ab1Qd565 was collected along the contour of the electrode at 2 V/1 kHz. Unlabeled NP (same product as above) was mixed in an equal amount of protein concentration with a liquid mixture of Ab1Qd565-carrying polyethylene fine particles and NPQSY9 under the same conditions as above and incubated for 5 minutes. While observing this using a fluorescence microscope, the alignment along the contour of the electrode at 2V/1 kHz was confirmed.

Fluorescence microscopy images during dielectrophoresis were electronically stored as still images on a connected computer regarding the presence/absence of unlabeled NPs. Comparing the two, it was confirmed that the fluorescence intensity was higher in the case with the presence of NP. These electronic images were loaded into Adobe Photoshop, and by cropping the images near the microelectrodes, erasing background noise, and emphasizing the contrast, it was confirmed that the differences between the two could be made clearer.

From the above results, it is thought that by creating dedicated software, these tasks can be performed consistently, and quantitative judgments can be made.

It was confirmed that when the inactivated virus (81N73, Hy Test) was applied to immunochromatography (anti-NP antibodies, FIA2121 and FIA3298, manufactured by Sino Biological) using a developing solution containing TritonX-100 (1%), the virus was destroyed during development, and NP appeared and caused a sandwich reaction with both antibodies, which could be detected by immunochromatography.

Judging from the above, it is considered that this Example made it possible to combine dielectrophoresis and fluorescence observation and demonstrated the possibility of detection with high sensitivity and high speed.

### Example 2

### Labeling of Antibody

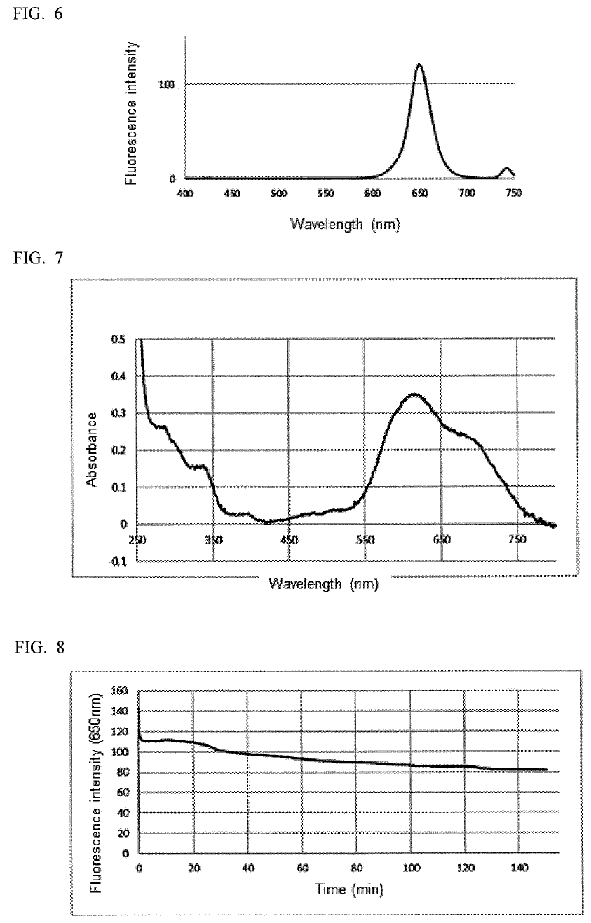

An anti-BSA mouse monoclonal antibody (Funakoshi Co., Ltd.) was used as an antibody that binds to BSA (Sigma). The antibody was concentrated to 100 μL (1 mg/mL) using the antibody concentrator included in the SiteClick (trademark) Qdot (trademark) 655 Antibody Labeling Kit (Invitrogen). According to the kit's method, sugar chains in the Fc region of the antibody were selectively chemically labeled with quantum dots (Qdot655, Thermo Fisher). The antibody Ab1Qd655 chemically labeled quantum dots was purified using the gel filtration device included in the kit. The fluorescence spectrum (excitation light: 370 nm) of the PBS solution measured using an Ab1Qd655 fluorescence spectrophotometer (F7100, Hitachi High-Tech Corporation) had peaks as shown in FIG. 6. The slightly visible peak at 720 nm is the scattering of twice the wavelength of the excitation light.

### Labeling of Antigen

QSY21 succinate ester (QSY21NHS, Thermo Fisher) was used as a quencher.

QSY21NHS is represented by the following formula. Although QSY21 has significant absorption near 650 nm, it is known to be a quencher that does not emit fluorescence, at least in the visible light region.

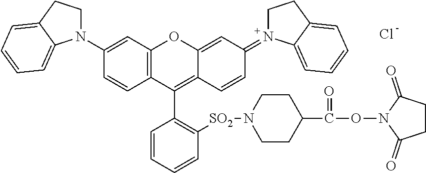

QSY21NHS was dissolved in anhydrous DMSO to a concentration of 10 mg/mL (12.3 mM). A DMSO solution of QSY21NHS was added to a 10 mM BSA solution (5 mg/mL) in an amount such that the number of molecules of BSA: QSY21NHS was 30 times the number of BSA molecules, and the mixture was stirred by vortexing. Further, an aqueous NaHCO₃ solution was added to a final concentration of 50 mM, and the mixture was left at room temperature for 3 hours. BSA labeled with a quencher was purified from the mixture using a spin column (BioRad P6). FIG. 7 shows the absorption spectrum of a solution obtained by diluting the purified product 5 times with PBS using a UV/VIS spectrophotometer (UV2500PC, SHIMADZU CORPORATION). When the number of bonds was calculated, there were 1.5 molecules of QSY21 per molecule of BSA.

### Confirmation Experiment on Quenching by FRET

Ab1Qd655 was diluted with PBS to a concentration of 10⁻⁸ M. The diluted solution was irradiated with 370 nm excitation light using a fluorescence spectrophotometer (F-7100, Hitachi High-Tech Science Corporation) using a fluorescence microcell (excitation-side optical path length: 10 mm; fluorescence-side optical path length: 2 mm, Hellma). The fluorescence spectrum from 350 nm to 800 nm was measured. Hereinafter, the wavelength range of the fluorescence spectra in the confirmation experiment on quenching was all the same.

1.0×10⁻⁶ M (1.0E⁻⁶M) BSAQSY21 was added to 10⁻⁸ M Ab1Qd655 to result in a final concentration of 10⁻⁸ M, and the mixture was stirred by vortexing for 5 seconds. After stirring, the mixture was quickly placed in a fluorescence microcell, and the fluorescence spectrum was measured using a fluorescence spectrophotometer.

The fluorescence spectrum was measured every 5 minutes for the next 30 minutes and every 10 minutes after 30 minutes until 150 minutes. FIG. 8 shows the results.

A 20% quenching occurred instantly after the start of the measurement, and a 72% decrease in fluorescence was eventually confirmed, although the rate was slow. In other words, a decrease in fluorescence due to fluorescence resonance energy transfer (FRET) based on antigen-antibody reaction was confirmed.

### Confirmation Experiment on Inhibition of Quenching

10⁻⁵ M unlabeled BSA was added to the reaction solution for the confirmation experiment on quenching to result in a final concentration of 10⁻⁷ M. After addition, stirring was quickly performed by pipetting. As in the confirmation experiment on quenching, the mixed solution was irradiated with 370 nm excitation light using a fluorescence spectrophotometer (F-7100, Hitachi High-Tech Corporation). The fluorescence spectrum from 350 nm to 800 nm was measured. Hereinafter, the wavelength range of the fluorescence spectra in the confirmation experiment on inhibition of quenching was all the same.

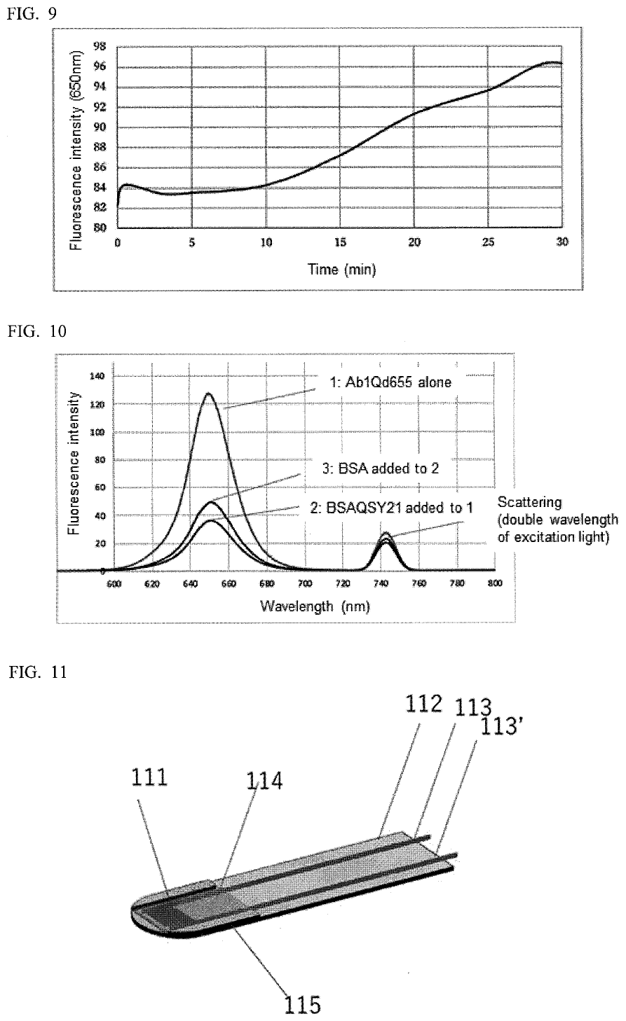

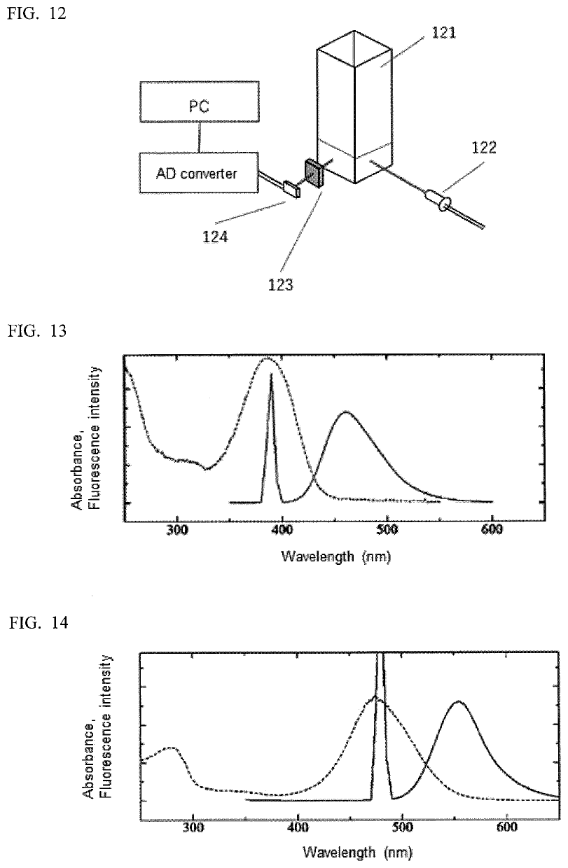

The fluorescence spectrum was then measured 3 minutes later, and thereafter, it was measured 12 times every 5 minutes. FIG. 9 shows the results.

The fluorescence, which was quenched by 72%, increased by 37% by adding unlabeled BSA. It was confirmed that Ab1Qd655 and BSAQSY21 were combined in an antigen-antibody reaction, and the quenched state was replaced by unlabeled BSA, increasing the fluorescence of Ab1Qd655. It was confirmed by observing the fluorescence enhancement that BSA could be detected without using B/F separation. FIG. 10 shows a comparison of the fluorescence spectra of the respective stages. When modified with Qd655, a solution of Ab1Qd655 tends to aggregate and precipitate during storage at 4° C. for several days. For example, this phenomenon is inhibited by arginine (from 10 mM to 500 mM, preferably from 50 mM to 200 mM).

### Dielectrophoresis

FIG. 11 shows a dielectrophoresis cell using a comb-shaped microelectrode manufactured by BAS. In FIG. 11, 111 denotes a microelectrode, in which opposite gold electrodes having an electrode length of 2 mm and an electrode width and an electrode spacing of 5 μm are printed on the substrate 112, and the electrodes are electrically connected to terminals 113 and 113′, respectively. A cover glass 114 was pasted on top of this via a 130-μm spacer 115, thereby forming a space (sample portion). A TEKTRONIX AFG3022 was used for high-frequency wave generation. One of the output terminals of AFG3022 was connected to terminal 113 via a 0.001-μF capacitor, thereby preventing direct current. An oscilloscope (Hewlett-Packard 54602B) was connected in parallel to terminals 113 and 113′ to monitor the waveform.

A sample in which yeast was dispersed in non-ionized water was introduced into the sample portion of the dielectrophoresis cell, and 2 MHz and 2 V were applied between terminals 3 and 3′ while recording images with a digital microscope (VHX900, 1000x, KEYENCE CORPORATION). It was observed that the dispersed yeast cells instantly aligned on the end surface of the electrode. Similar results were observed at frequencies of 5 MHz, 1 MHz, 300 kHz, and 100 kHz.

No dielectrophoresis was observed for any of the related particles, which were indium tin oxide (ITO) and antimony tin oxide (ATO) having a particle size of 2 μm and poly beads (gold-plated on the surface of polystyrene particles) type A (MB-A) and type B (MB-B) manufactured by SEKISUI CHEMICAL CO., LTD. However, after these were immersed in a PBS solution containing 0.1% BSA, centrifuged at 5000 g for 20 minutes after 8 hours, and the supernatant was discarded twice to coat the surface with BSA, each of ITO, ATO, MB-A, and MB-B aligned instantly at 2 MHz, 5V, in 1 mM PBS. In other words, concentration occurred at the electrode end.

For graphite fine particles (Ito Graphite Co., Ltd.), very fast dielectrophoresis could be observed at 2 MHz, 5 MHz, and 300 kHz in 1 mM PBS, regardless of whether they were coated with BSA. Dielectrophoresis was confirmed even when the concentration of PBS was set to 10 mM, although a slight decrease in speed was observed. Each case of dielectrophoresis described above was positive dielectrophoresis that gathering was observed at the electrode ends (more precisely, at the corners of the printed electrode). For reference, the conductivity of PBS at various concentrations was measured using a conductivity meter (Laqua Twin EC-33) manufactured by HORIBA, Ltd.: 10 mM: 13,000 μS/cm; 2 mM: 3,360 μS/cm; 1 mM: 2,110 μS/cm. It is 2,000-8,000 μS/cm for saliva, as written in a reference (Yen-Pei Lu et al., Scientific Reports 1(2019) 9:14771), and the actual value measured using real specimens was 5,780 μS/cm.

### Dedicated Measurement Device

FIG. 11 shows the basic configuration of a measurement unit. The detection cell 121 is a four-sided transparent fluorescence measurement cell having an optical path length of 10 mm, and a color filter 123 (sharp cut filter Y50, HOYA) completely blocks light having wavelengths shorter than 500 nm. LED 122 is OSV2YL5111A (manufactured by Opto Supply) and emits 370 nm of light. The excitation wavelength used was around 370 nm. The sensor 124 is S7183 (Hamamatsu Photonics K.K.) and has a center of sensitivity around 650 nm.

The AD converter used was Arduino Uno, and the program was created with Arduino IDE. In addition, a PC and Arduino were connected via USB, and the program on the PC was created using Processing.

The steps of operation are shown below.
The surface of the detection cell is blocked in advance using a protein other than BSA to prevent non-specific adsorption.
PBS solutions of Ab1Qd655 and BSAQSY21 (10⁻⁸ M each) are mixed and incubated at room temperature for 24 hours.
The resulting liquid mixture is placed into the detection cell and the application is launched on the PC.
Instructions are sent from the software on the PC to allow the AD converter to measure the output voltage of the sensor, perform AD conversion, and send the signals to the PC via the USB interface. This is performed every 0.5 seconds five times in total, and the average value is saved as the initial value.
The message “Ready” is displayed on the PC monitor.
The tester adds the sample to the detection cell (at this time, the volume change should be within a negligible range).
A vibrator (not shown) in contact with the detection cell vibrates and stirs the liquid for 3 minutes.
The sensor signal is read and sent to the PC. This is repeated five times, and the PC calculates the average value and saves it as a measured value.
Stirring by the vibrator is stopped.
The PC compares the initial and measured values, and if the fluorescence intensity increases by more than a predetermined threshold (for example, 5%), it displays a “positive” determination. At this time, as a reference value, the percentage of increase is displayed, and in addition to the two levels of “positive” and “negative,” semi-quantitative values may be displayed in from 5 to 10 levels.

By simply mixing the sample, it is possible to qualitatively or quantitatively detect the target object in a homogeneous solution without performing a development process like a lateral flow method, a washing process like ELISA, or other B/F separation.

### Example 3

When Ab2 and Ab3 are labeled with different fluorescent dyes (F2, F3) in a so-called sandwich-type antigen-antibody reaction in which two types of antibodies (Ab2 and Ab3) bind to one antigen simultaneously, provided that F3 is excited by the fluorescence emitted by F2 (wavelength λF2) and emits wavelength λF3, but F3 is not excited by the excitation light of F2 (wavelength λE2), it is possible to monitor the progress of the sandwich reaction by causing λE2 excitation and detecting the fluorescence intensity at wavelength λF3.

An example in which two types of antibodies were prepared to target the nucleoprotein (NP) of the influenza A virus will be described below.

At the same time, antibodies FIA2121 and FIA3298 (both anti-NP mouse monoclonal antibodies manufactured by BIO MATRIX RESEARCH, INC.) were used as antibodies that form an antibody-antigen conjugate of Ab2, NP, and Ab3 as a result of the sandwich reaction with NP.

A labeling reaction was performed using the antibody FIA2121 as Ab2 and 10 μL of a PBS solution (1 mg/mL) as a starting point according to the protocol disclosed by ATTO390NHS Ester (ATTO-Tec). The reaction solution was purified using Nanosep (registered trademark) with a cut-off molecular weight of 30 k. The number of pigment molecules bound per antibody molecule calculated from the absorption spectrum was 23. FIG. 13 shows the absorption spectrum (broken line) and the fluorescence spectrum (solid line) upon excitation at 390 nm of the obtained labeled antibody (Ab2AT390).

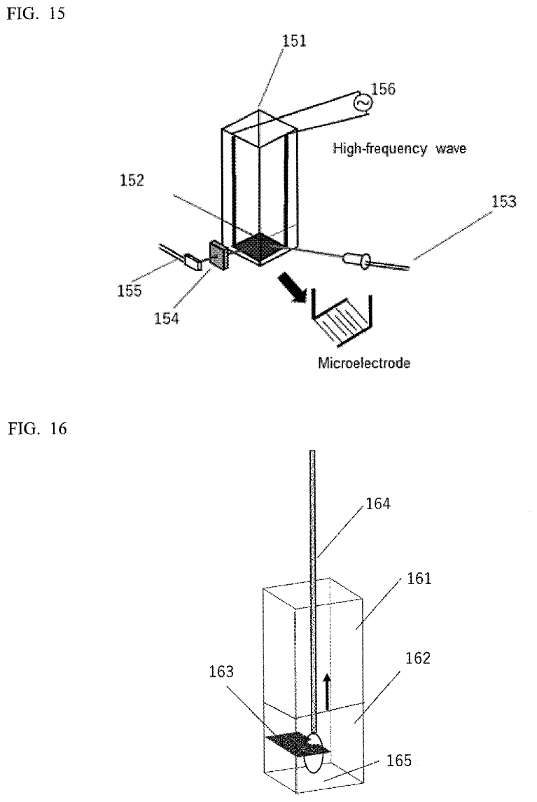

The antibody FTA3298 was used as Ab3 to prepare 100 μL of a PBS solution (2 mg/mL), 20 μL of a 1M NaHCO₃ aqueous solution was added, and then immediately 25 μL of a DMSO solution (2 mM) of the dye DY485XLNHS ester (Dyomic) (37.5 times the number of molecules of the antibody) was added, and stirred well. After 5 minutes, the reaction solution was purified using Nanosep (registered trademark) with a cut-off molecular weight of 30 k (Nippon Genetics). The number of pigment molecules bound per antibody molecule calculated from the absorption spectrum was 37. FIG. 14 shows the absorption spectrum (broken line) and the fluorescence spectrum (solid line) upon excitation at 470 nm of the obtained labeled antibody (Ab3DY485).

From the results of FIGS. 13 and 14, it is understood that when a reaction solution containing two types of labeled antibodies is irradiated at 390 nm, only Ab2AT390 is excited and emits fluorescence around 470 nm, while Ab3DY485 is not excited because the molecules of Ab2AT390 and Ab3DY485 are sufficiently spaced apart. In addition, when NPs are present in the solution, Ab2AT390 and Ab3DY485 molecules bind to the NPs at a short distance, estimated from 5 to 10 nm, resulting in energy transfer by FRET. In other words, the excitation energy corresponding to the fluorescence around 470 nm emitted by Ab2AT390 is transferred, and Ab3DY485 is excited and caused to emit fluorescence at 560 nm. This indicates that the presence of NP can be determined by exciting the reaction solution at 390 nm and observing fluorescence at 560 nm.

From the results of Examples 1 to 3, the present invention can be implemented by the following embodiments. The following embodiments are shown as illustrations and do not limit the scope of the present invention. The present invention can be implemented with various modifications.

### Embodiment 1

Monoclonal antibodies mAb11, mAb12, and mAb13, which have three different epitopes in the nucleoprotein (NP) of influenza A virus, are used.

ITO, having an average particle size of 2 μL, is dispersed in advance at a concentration of 0.2 mg/mL in 1 mL of a 10 μg/mL solution of mAb11. Incubation is performed for 2 h with thorough stirring, followed by centrifugation at 5000 g/5 min. The supernatant is discarded. The precipitate is dispersed in a 1 mg/mL BSA solution (containing 0.05% sodium azide and 1% TritonX-100), thereby preparing a reagent 11.

Meanwhile, mAb12 and mAb13 are labeled with ATTO390 and DY485XL, respectively, thereby obtaining labeled antibodies Ab12AT390 and Ab13DY485. A 1 mM PBS (containing 0.05% sodium azide) containing 10⁻⁷ M of Ab12AT390 and Ab13DY485 is prepared and used as a reagent 12.

As a specimen, 100 μL of saliva containing inactivated influenza A virus is mixed with 400 μL of the reagent 11 and left for 5 minutes. At this time, the viral envelope is destroyed by TritonX-100, and NP is released (confirmed by evaluation using in-house immunochromatography). The released NP binds to mAb11 adsorbed on the ITO surface. Then, the resulting product is mixed with 500 μL of the reagent 12. At this point, the saliva has been diluted 10 times, and the conductivity has dropped to below 1000 μS/cm, at which sufficient dielectrophoresis may occur.

The mixture is introduced into a detection cell provided with a microelectrode on the bottom. Detection is performed in the following steps.
1) An AC voltage of 2 MHz, 5 V, is applied to the microelectrode.
2) The area around the microelectrode is irradiated with a 390 nm light source, and the fluorescence intensity around 560 nm is observed.
3) Fluorescence observation is performed 10 times sequentially, and the average value is sent to the PC.
4) This is performed every 5 seconds for a total of 2 minutes, and the final intensity is calculated from the tendency of increase in fluorescence intensity.
5) A negative/positive determination is made based on the final intensity and quantitative data.

In Embodiment 1, by binding NP derived from a specimen to ITO via mAb11, the NP concentration is locally increased near the microelectrode by dielectrophoresis. Concentration on the electrode surface (XY plane) is expected to be about 100 times. In the depth direction (Z-axis), the frequency of forced contact with the electrode surface can be increased by efficiently performing convection in the depth direction of the cell using a vibrator. Therefore, it becomes possible to concentrate the specimen by 1000 times or more in total.

In Embodiment 1, for example, a fluorescent dye can be applied as long as it has a high molecular extinction coefficient and a high quantum yield, and it is a combination of dyes that efficiently cause the FRET phenomenon. Although reagents 11 and 12 are prepared as different reagents to make the reaction easier to understand, they can be mixed in advance. Further, it is obvious that when reagents 11 and 12 are prepared by adhering them to a detection cell and freeze-drying them, the number of steps during testing can be further reduced. Similarly, the above can also be appropriately selected and applied in Embodiments 2 and 3.

### Embodiment 2

Monoclonal antibodies mAb21 and mAb22, which have two different epitopes in the nucleoprotein (NP) of influenza A virus, are used. In addition, a fragment of NP (NP fragment) that binds to mAb22 is used.

Since NP can be synthesized by genetic recombination, mAb21 and mAb22 may be prepared by first determining a fragment of NP and then producing antibodies using the NP fragment as an immunogen. It is necessary to confirm that the obtained antibody recognizes NP. At this time, the reaction rate constant (ka: association rate constant; kd: dissociation rate constant) between mAb22 and the NP fragment can be determined, for example, by the surface plasmon resonance method. Desirably, antibodies with ka>10⁶ and kd<10⁻³ can be screened to achieve high measurement speed.

mAb21 is adsorbed to the ITO surface, and the ITO is then blocked with BSA, thereby preparing a reagent 21 containing 1% TritonX100.

Meanwhile, mAb22 is labeled with Qd655, and the NP fragment is labeled with QSY21. A 1 mM PBS solution (containing 0.05% sodium azide) containing the labeled antibody Qd655Ab22 and the labeled antigen fragment QSY21NP at a concentration of 10⁻⁸ M is prepared as a reagent 22. Testing is performed in the following steps.
1) A specimen (saliva containing inactivated influenza A virus) in an amount of 100 μL is mixed with 400 μL of the reagent 21 and left for 5 minutes.
2) The resulting liquid mixture is mixed with 500 μL of the reagent 22.
3) The liquid mixture is immediately introduced into a detection cell provided with a microelectrode on the bottom.
4) Stirring is started with a vibrator. The vicinity of the microelectrode is irradiated with a wavelength of 370 nm or less, and the fluorescence emitted from the vicinity of the microelectrode is measured by a sensor through a color filter that cuts wavelengths of 500 nm or less.
5) The first measured value is saved as an initial value, subsequent changes in fluorescence intensity are measured, and the final measured value is calculated from the trend of the changes.
6) In advance, a state is created in which Qd655Ab22 and ASY21NP bind to each other, and a certain degree of quenching occurs. From such a state, when Qd655Ab22 binds to the NP present near the electrode, the presence or absence of NP and whether the infection is positive or negative are determined based on inhibition of quenching (i.e., enhancement of fluorescence).

### Embodiment 3

Ab31 and Ab32, which simultaneously bind to the influenza A virus surface antigen, are used. Ab31 and Ab32 are labeled with ATTO390 and DY485XL, respectively (hereinafter referred to as Ab31AT390 and Ab32DY485). A 1 mM PBS (containing 0.05% sodium azide) containing at least 10⁻⁷M of these is prepared (reagent 31). Testing is performed in the following steps.
1) The specimen (saliva containing influenza A virus) in an amount of 100 μL is mixed with 900 μL of the reagent 31 and introduced into a detection cell provided with a microelectrode on the bottom.
2) A high-frequency wave of from 1 kHz to 5 MHz, preferably 100 kHz, and 5 V is immediately applied to the microelectrode, and stirring is started using a vibrator. 3) The vicinity of the electrode is excited at 390 nm, and the fluorescence intensity, which is around 560 nm, is measured.
4) The final measured value is calculated from the time-dependent changes in fluorescence intensity.
5) The virus concentration and the infection status are determined from the final measured value.

In Embodiment 3, The virus itself is concentrated in the vicinity of the electrode by dielectrophoresis. Therefore, a surfactant and an incubation time required for NP extraction are unnecessary.

### Embodiment 4

A detection cell 151 is used, which is provided with a microelectrode in which comb-shaped electrodes having an electrode width and an electrode spacing of 5 μm and an electrode length of 5 mm face each other on the bottom surface. An LED light source 153 and a sensor 155 are tilted toward the microelectrode. Thus, a detection device is composed (FIG. 15). The analog signal output by the sensor 155 is processed.

ITO, having a particle size of 2 μm, is dispersed at 0.11 mg/mL in a PBS solution containing 10⁻⁶ M of BSAQSY21 and left overnight at room temperature, followed by centrifugation at 5000 g for 20 minutes. Then, the supernatant is discarded. Next, the resulting product is dispersed in skim milk with a 1 mg/mL concentration and blocked, followed by centrifugation. Then, the supernatant is discarded, and the precipitate is collected. A PBS dispersion solution of Ab1Qd655 (final concentration: 10⁻⁸ M) with BSAQSY21 is prepared to result in a final BSAQSY21 concentration of 10⁻⁸ M, assuming a loss due to centrifugation of 30%. The PBS dispersion solution is left at room temperature overnight. BSA is measured using the following steps.
The obtained mixed dispersion solution is introduced into the detection cell, and the application is launched on the PC.
Instructions are sent from the software on the PC to allow the AD converter to measure the output voltage of the sensor at the same time as the LED emits 370 nm, perform AD conversion, and send the signals to the PC via the USB interface. This is performed every 0.5 seconds five times in total, and the average value is saved as the initial value.
The message “Ready” is displayed on the PC monitor.
The tester adds the sample to the detection cell (at this time, the volume change should be within a negligible range).
An oscillator (not shown) in contact with the detection cell oscillates and stirs the liquid for 3 minutes. At the same time, a high-frequency wave of 5 V, 2 MHz is applied between the microelectrodes.
The sensor signal is read and sent to the PC. This is repeated five times, and the PC calculates the average value and saves it as a measured value.
Stirring by the vibrator and applying a high-frequency wave are stopped.
The PC compares the initial and measured values, and if the fluorescence intensity increases by more than a predetermined threshold (for example, 5%), it displays a “positive” determination. At this time, as a reference value, the percentage of increase is displayed, and in addition to the two levels of “positive” and “negative,” pseudo-quantitative values may be displayed in from 5 to 10 levels.

In Embodiment 4, a preliminarily prepared thing that is equivalent to an object to be detected but not an object to be detected (pseudo-antigen) is adsorbed on the carrier surface such that the pseudo-antigen is concentrated in the vicinity of the electrode by dielectrophoresis. Similarly, it is also possible to bind a labeled antibody to the surface of a carrier and concentrate it by dielectrophoresis. The concentration of antigen-antibody conjugate in antigen-antibody reaction is proportional to the fee antibody concentration, free antigen concentration, and body affinity. Therefore, as described herein, in a case in which the pseudo-antigen competes with the antigen, specific effects can be obtained even after allowing any of the antibody, pseudo-antigen, or detection substance to be adsorbed by the carrier and concentrated by dielectrophoresis. By concentrating the substance to be detected in the specimen, it is desirable to improve the sensitivity of the system most efficiently, even when using an antibody with the same affinity.

### Embodiment 5

As a specimen, 100 μL of saliva is collected. Although the collection method is arbitrary, a cotton swab 164 is effective in providing a certain degree of quantitative performance without the use of a particular instrument (FIG. 16). A reaction solution is contained in advance at a level of 162 in a test container 161 provided with a microelectrode 165. A swab 164 is hooked into the indentation of a throttle plate 163 to be squeezed such that the specimen is mixed with the reaction solution.

As the reaction solution, 1% TritonX-100 dissolved in 1 mM PBS is mixed in advance with RNA1 and RNA2 complementary to base sequences at different positions in influenza A RNA as materials for recognizing the substance to be detected. RNA1 is a pyrene-modified 2′-O-methyl oligo RNA with reference to Akira Murakami et al., Kagaku to Seibutsu (KASEAA) Vol. 41, No. 8, 2003. RNA2 was physically adsorbed onto graphite with a particle size of 2 μm and then blocked with 0.1% BSA. The following steps are performed when the reaction solution described above is installed in a detector.
1) According to a control signal sent from a PC, a vibrator in contact with a test container is activated, and stirring by convection begins.
2) In a case in which the specimen contains the influenza A virus as a substance to be detected, as a result of stirring for 30 seconds at room temperature, the action of TritonX-100 destroys the virus envelope, and single-stranded RNA is exposed to the reaction solution. This RNA is bound to RNA1 and RNA2 as materials for recognizing the substance to be detected (RNA-binding product).
3) A signal sent from the PC applies a high-frequency wave of 2 MHz, 5V, to the microelectrode placed on the bottom of the detection container. This concentrates the RNA-binding product in the XY direction of the bottom surface along the microelectrode. Further, convective stirring allows the reaction solution to contact the microelectrode evenly, thereby achieving concentration in the Z direction.
4) An LED (STANLEY ELECTRIC CO., LTD., MNU1109EAE-275) irradiates 275 nm light near the microelectrode.
5) A color filter cuts out light with a wavelength of 350 nm or less, and a light sensor (HIOKI E.E. CORPORATION, 9743) detects the 380 nm fluorescence that passes through the color filter. After AD conversion, a digital signal is sent to the PC.
6) The PC records the output fluctuations of the light sensor over 3 minutes, evaluates the difference from the value detected in advance with a negative control (buffer adjusted to a conductivity of 8000 μS/cm), and displays determination results or semi-quantitative data on the presence or absence of influenza virus.

A carrier is used herein to ensure dielectrophoresis. As long as the conductivity of the reaction solution is sufficiently low to allow an increase in applied voltage and a decrease in detection speed, the system may be further simplified by directly concentrating RNA by dielectrophoresis without a carrier.

Embodiment 5 describes a case in which the fluorescence labeled on RNA1 is enhanced due to the formation of a double strand. In Embodiment 6, which is a modified version of Embodiment 5, RNA11, which is complementary to a partial base sequence of influenza A RNA and immobilized on a carrier, and RNA12, which is complementary to RNA11, are labeled with fluorescence and a quencher, respectively. When RNA11 and RNA12 are bound, quenching usually occurs. However, it is also possible to detect that RNA11 binds to a substance to be detected such that quenching is inhibited when the influenza A RNA as a substance to be detected is further mixed. In this case, as long as RNA11 is made sufficiently longer than RNA12 such that the affinity between RNA11 and the substance to be detected increases, the binding between RNA11 and the substance to be detected takes priority over the binding between RNA11 and RNA12, which is preferable from both the reaction rate and sensitivity. Similarly, RNA21 complementary to a part of the base sequence of influenza A RNA and immobilized on a carrier as a material for recognizing the substance to be detected, a fluorescent label at the 5′ end of RNA21, and a molecular probe labeled with a quencher probe at the 3′ end of RNA21 available as a molecular beacon from Merck or Primetech Corporation may be used. Although the above description has been made using RNA as the material for recognizing the substance to be detected, the substance to be detected may be RNA, and the material for recognizing the substance to be detected may be DNA complementary to the substance to be detected.

### INDUSTRIAL APPLICABILITY

The detection method of the present invention is useful in testing for infectious diseases that were conventionally undetectable, in on-site testing where conventional testing was necessary but could not be performed, and in testing for items that could not be tested in principle with genetic testing and the like.

### REFERENCE SIGNS LIST

41 Substrate
42 Microelectrode
43 Spacer
44 Cover
45 Capillary
46 Air hole
47 Electrode 1
48 Electrode 2
4A Direction of sample introduction
4B Enlarged view of microelectrode
61 Detection cell
62 Oscillator
63 Light source
64 Excitation light
65 Lens
66 Fluorescence
67 Light filter
68 Light sensor
69 Terminal
111 Microelectrode
112 Substrate
113, 113′ Terminal
114 Cover glass
115 Spacer
121 Detection cell
122 LED
123 Color filter
124 Sensor
151 Detection cell
152 Microelectrode
153 LED
154 Color filter
155 Sensor
156 High-frequency wave
161 Test container
162 Reaction solution
163 Throttle plate
164 Swab
165 Microelectrode

1. A method for detecting a substance to be detected within a sample, comprising:
concentrating locally the substance to be detected, a material for recognizing the substance to be detected, or a binding product of the substance to be detected and a material for recognizing the substance to be detected within a sample by electrophoresis or dielectrophoresis;
mixing the substance to be detected, a material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence at the specific wavelength in the sample to bind the substance to be detected and the material for recognizing the substance to be detected, or
mixing the substance to be detected, a material for recognizing the substance to be detected which is labeled with a fluorescent substance that emits fluorescence at a specific wavelength, and the substance to be detected or a substance other than the substance to be detected which is recognized at the same site as the substance to be detected for the material for recognizing the substance to be detected, which is labeled with a fluorescent substance that fluoresces at a wavelength different from the specific wavelength or a quencher that absorbs fluorescence with the specific wavelength in the sample to bind the substance to be detected and the material for recognizing the substance to be detected;
measuring changes in fluorescence intensity of a binding product of the substance to be detected and the material for recognizing the substance to be detected; and
confirming the presence of the substance to be detected in the sample based on the measured fluorescence intensity,
wherein the material for recognizing the substance to be detected is labeled with a fluorescent substance that emits fluorescence at a specific wavelength and specifically recognizes and binds to the substance to be detected, and
measuring changes in fluorescence intensity includes
inducing excitation at wavelength 1 so as to measure a fluorescence intensity of wavelength 3, wherein the fluorescent substance that emits fluorescence at the specific wavelength is a fluorescent substance that is excited at wavelength 1 and emits wavelength 2, and the fluorescent substance that fluoresces at a wavelength different from the specific wavelength is a fluorescent substance that absorbs wavelength 2 and emits wavelength 3, or
inducing excitation at wavelength 1 so as to measure a fluorescence intensity of wavelength 2, wherein the fluorescent substance that emits fluorescence at the specific wavelength is a fluorescent substance that is excited at wavelength 1 and emits wavelength 2, and the quencher that absorbs fluorescence with the specific wavelength is a fluorescent substance that absorbs wavelength 2 but does not emit fluorescent at least in a measurement wavelength range.

2. (canceled)

3. (canceled)

4. The method according to claim 1, wherein the material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength simultaneously, bind to the substance to be detected in the sample.

5. (canceled)

6. (canceled)

7. The method according to claim 1, wherein any one selected from the group consisting of the material for recognizing the substance to be detected, the substance to be detected, and the substance other than the substance to be detected, which is recognized at the same site as the substance to be detected, for the material for recognizing the substance to be detected, is physically or chemically bound to a carrier particle.

8. The method according to claim 1, wherein the material for recognizing the substance to be detected which is labeled with a fluorescent substance that emits fluorescence at a specific wavelength, and a material for recognizing the substance to be detected which is labeled with a fluorescent substance that fluoresces at a wavelength different from the specific wavelength or a quencher that absorbs fluorescence with the specific wavelength, simultaneously bind to the substance to be detected in the sample, and
wherein a material for recognizing the substance to be detected, which is physically or chemically bound to the carrier particle, is further contained, and the substance to be detected and the material for recognizing the substance to be detected form a binding product in the sample.

9. The method according to claim 7, wherein the carrier particle is a metal fine particle, a metal oxide fine particle, a non-metal fine particle, a metal-coated resin fine particle, or a non-infectious spherical biological fine particle.

10. The method according to claim 1, wherein locally concentrating is carried out by positive dielectrophoresis.

11. The method according to claim 1, wherein the material for recognizing the substance to be detected is an antibody or an antibody fragment.

12. The method according to claim 1, wherein the substance to be detected is a bacterium, a virus, a nucleic acid, a protein, or a peptide.

13. The method according to claim 1, wherein the substance to be detected is an influenza-virus-derived substance.

14. The method according to claim 1, wherein at least one fluorescent substance is a quantum dot.

15. The method according to claim 1, wherein the substance to be detected is a nucleic acid, and the material for recognizing the substance to be detected is a nucleic acid complementary to at least part of the nucleic acid as the substance to be detected.

16. The method according to claim 15, wherein the substance to be detected is single-stranded RNA.

17. The method according to claim 8, wherein the carrier particle is a metal fine particle, a metal oxide fine particle, a non-metal fine particle, a metal-coated resin fine particle, or a non-infectious spherical biological fine particle.
